SEQUENCE LISTING (4 sequences)

SEQ ID NO: 1 - protein, 6 aa, Artificial Sequence
  Peptide which can be cleaved by cathepsin K.
QCGKPG

SEQ ID NO: 2 - protein, 6 aa, Artificial Sequence
  Peptide which can be cleaved by ADAMTS-4.
KCEFVG

SEQ ID NO: 3 - protein, 10 aa, Artificial Sequence
  Peptide which can be cleaved by ADAMTS-4.
GVQEFRGVTG

SEQ ID NO: 4 - protein, 10 aa, Artificial Sequence
  Peptide which can be cleaved by MMP-13.
GPLGVRGKGG

(19) United States
(12) Patent Application Publication
(10) Pub. No.: US 2015/0329592 A1
(43) Pub. Date: Nov. 19, 2015

(54) ALL GOLD FLUORESCENCE RESONANCE ENERGY TRANSFER PROBE AND USE THEREOF

(71) Applicant: KAOHSIUNG MEDICAL UNIVERSITY, Kaohsiung City (TW)

(72) Inventors: Chih-Kuang Wang, Kaohsiung City (TW); Mei-Ling Ho, Kaohsiung City (TW); Chung-Hwan Chen, Tainan City (TW); Yin-Chih Fu, Kaohsiung City (TW); Jia-Huie Lin, Kaohsiung City (TW); Chih-Yun Lee, Chiayi County (TW); Je-Ken Chang, Kaohsiung City (TW)

(73) Assignee: KAOHSIUNG MEDICAL UNIVERSITY, Kaohsiung City (TW)

(21) Appl. No.: 14/708,305

(22) Filed: May 11, 2015

(30) Foreign Application Priority Data
May 16, 2014 (TW) 103117242

(51) Int. Cl. C07K 7/06

(52) U.S. Cl. CPC C07K 7/06

(57) ABSTRACT

The present invention relates to a gold fluorescence resonance energy transfer nanoprobe comprising a gold fluorescence donor, a gold fluorescence acceptor, and a linker fragment that connects the gold fluorescence donor and the gold fluorescence acceptor, wherein the fluorescence resonance energy transfer is carried out between the gold fluorescence donor and the gold fluorescence acceptor. This all gold probe employing fluorescence resonance energy transfer technique can be used for detecting diseases such as arthritis, osteoporosis, and cancer metastasis.

### CROSS-REFERENCES TO RELATED APPLICATIONS

The present application claims priority to Taiwan Patent Application No. 103117242 filed on May 16, 2014, incorporated herein by reference in its entirely. The sequence listing text file, file name 2376-KMU-USSEQLIST_ST25 created May 8, 2015, file size 1245 bytes, is incorporated herein by reference in its entirely.

### FIELD OF THE INVENTION

The present invention relates to an all gold probe utilizing fluorescence resonance energy transfer technique, especially relates to a use of the all gold probe in detecting diseases.

### BACKGROUND OF THE INVENTION

Nowadays, human's quality of life and the medical techniques are getting much better than used to be, leading to longer human lifespan and an increase of elderly population, in which the phenomenon is especially significant in developed countries. According to WHO analysis, osteoarthritis is in the top 5 of common diseases among elder people. There are multi-pathways that may lead to the same symptoms of osteoarthritis, making osteoarthritis a complex disease. The initial symptoms of this chronic disease are not quite notable, however, as the disease getting worse, the stiffness and pain gradually appear, and then cause limited mobility. In the conventional treatment, taking anti-inflammatory medicine can merely reduce painful, rather than slow the deterioration of osteoarthritis; at the later stage of the disease, patients can just replace their joints with artificial joints by surgeries. It can not only extend the lifetime of elders' articular cartilages but also significantly decrease the related medical costs, if we can detect osteoarthritis in the early stage and use drugs to inhibit the production of inflammatory substances and restriction enzyme to reduce the damages of cartilage cells, thus regulating the progression of arthritis.

Arthritis is a generic term, it usually refers to an inflammation occurring in joints, and there are abrasions and lacerations appearing on the surface of articular cartilages. Osteoarthritis is one of the most common types of arthritis as it may occur at any joints, especially at joints that bear more weight or are more frequently used, e.g. hip joints, knees, spinal joints, and fingers etc. The progression of osteoarthritis includes lesions on the surface of cartilage, degradation and loss of glycosaminoglycan molecules (e.g. hyaluronic acid) which may be accompanied by cartilage sclerosis and followed by the production of osteophyte, the influence of which then causes damages to peripheral synovium, ligament, and muscle. The biggest difference between osteoarthritis and another common arthritis “rheumatoid arthritis” is that osteoarthritis only occurs at joints and doesn't cause any other inflammation or damage in other organs, while rheumatoid arthritis is an autoimmune disease which not only causes inflammation in the tissues around the joints but also causes inflammation and damage in other organs. Arthritis brings joint stiff and aches to patients, thus causes inconvenience in daily life. The present treatment cannot stop the disease progression from exacerbation, and the cost of the treatment is also considerable. Under the current changes of life quality and medical technology, the prolongation of life is inevitable, which makes osteoarthritis one of the most common problems. Hence, one should place importance on osteoarthritis.

Articular cartilage is mainly constructed by extracellular matrix (ECM). Articular cartilage is divided into four zones according to its functional biochemical characteristics: (from outside to inside) superficial zone, middle zone, deep zone, and calcified zone. The contents of the structures are: 70-75% of water, 10-15% of collagen (mainly is type II collagen, others are types V, VI, IX, X and XI collagen), and the rest of the contents are proteoglycan, non-collagen, and chondrocytes that account for the least. Every element has its own role in maintaining the basic function of joint. The type II collagen mainly forms the net structure, which provides support for articular cartilage. Proteoglycan is a structural protein, bearing a great quantity of negative electric charge that makes it possible to absorb a big amount of water. It has a plumose appearance, formed by hyaluronic acid as trunk and many of proteoglycan chains. Under normal circumstances, the articular cartilage has a balanced synthesis and metabolic system. The chondrocytes synthesize matrix, many kinds of chondrolysis enzymes (e.g. proteoglycan lytic enzyme, matrix metalloprotease, and cysteine proteinase etc.), enzymatic activity inhibitor, and cytokines (e.g. tumor necrosis factor-α (TNF-α), and interleukin-1 (IL-1)) that regulate articular cartilage activity, thereby metabolize cartilage and growth factors to promote the synthesis of proteoglycan and the growth of chondrocytes, thus maintain the kinetic balance in metabolism.

Many pathways could lead to osteoarthritis, making this a complex disease. The main causes of osteoarthritis are: (1) primary osteoarthritis: it is common in older people but the pathogenesis still remains unknown, which may be related to age, perennial loaded-working, genes, overweight, hormones etc. or other factors unknown; (2) secondary osteoarthritis: it's caused by other known factors, e.g. injury to joints, bacterial infections, or repetitive joint use (e.g. athletes), and the secondary osteoarthritis generally occurs in younger people.

The articular cartilage is composed mostly of type II collage, forming a reticular formation to maintain joint functions. When type II collagen loses, the articular cartilages transforms into other types of collagen to reduce the loss. However, other types of collagen cannot afford joint compress and stretch; hence the ability of articular cartilages to withstand pressure will decrease, and thus causes damages to the surface of articular cartilage. It is presumed that the mechanism may go through two pathways: (1) synovial fibroblasts may release cytokines to activate proteases, and directly cause damage to the cartilage; (2) cytokines promote abnormal rapid growth of chondrocyte, leading to ossification and producing proteases for destruction. The proteases are grouped into three categories. (1) Matrix metalloproteinase (MMP), a neutral endopeptidase, pH value 6.5 to 8, which has its specific cutting site on type II collagen. The major MMP found in articular cartilage is MMP-13; (2) cathepsin K, a cysteine protease, is found having a wide range of cutting sites, and it mainly cuts type II collagen on many cutting sites; cathepsin K is also found that it can cleave type I collagen, which may take the place of type II collagen; in 2012, it was also found to cleave the proteoglycan sites; (3) aggrecanase, which mainly works on proteoglycan, and the most common types in osteoarthritis research are ADAMTS-4 and ADAMTS-5.

It's a breakthrough in modern medicine to introduce nanotechnology into medical area, using nanomaterial as human molecular tools to do disease diagnosis, disease prevention, and drug delivery, etc. However, the size of nanomaterial is much smaller so that it may easily enter bio-tissue or bio-cells; thus, the biocompatibility and bio-toxicity of the material, which may cause cell apoptosis, should not be neglected when applied. Currently common biomedical nanoparticles include metal nanoparticles (e.g. gold, silver, iron . . . etc.), macromolecule nanoparticle, carbon nanocluster, etc. Recent years, nanometal is broadly used in biomedicine, because that the material itself has certain unique features, e.g. optical, electrical, magnetic, and thermal properties, leading continued breakthroughs in disease diagnosis and treatment. So far there are the most abundant researches in gold nanoparticle.

The most commonly used gold nanoparticle is gold nanorod (GNR). Since GNR are different in shape, length, and width, GNRs can obtain a variety of absorption peak spectrums due to different aspect ratio. The absorption peak of long axis of GNRs having larger aspect ratio will shift to near infrared region. The absorption spectrum of GNR in near infrared region indicates that the GNR could absorb the energy in near infrared region, making the GNR one of the best choices of nanoparticles to be used in biomedical materials. The light with wave band in near infrared region, when being applied in organisms, not only can prevent harm caused by light absorption in tissue but also can penetrate into deeper tissues to achieve diagnosis and treatment in the deeper tissue.

Recent years, Fluorescence Resonance Energy Transfer (FRET) technology has been widely used in nanomaterial as disease detector, drug release system, etc. One end of the material is designed as energy donor (e.g. fluorescence material), and another end is designed as energy acceptor (e.g. GNR); when the fluorescence material links to GNR, the emitted fluorescence range is exactly in the range absorbable by GNR, so that the system is then established. The fluorescence energy emitted from fluorescent material will transfer to GNR, thus no fluorescence appears; yet the fluorescence appears under the condition that the fluorescent material and the GNR are separate in a distance that is longer than a valid energy transfer distance. This system is quite suitable for being used in disease diagnosis and treatment.

Semiconductor quantum dot is a nanoscale semiconductor material, constituted by a small number of atoms, with size of about 2-20 nm. In this extremely small size, the interior of the quantum dot has a three-dimensional energy barrier, which limits the internal electrons activities, and thus causes quantum confinement effect. Since the quantum confinement effect would make the material produce a discrete electronic state structure which is similar to atoms, the materials will produce different emission light based on different sizes of the particle. The valid energy band-gap of semiconductor nanoparticle increases as the radius of the particle decreases. Therefore, the larger the particle size is, the closer to near infrared region the emission light shifts to; on the other hand, the smaller the particle size is, the closer to blue light region the emission light shifts to. The present semiconductors have cores made by CdSe and CdTe, which are coated by ZnS, ZnSe, and CdS as core shell to obtain higher light-emitting efficiency and better stability. However, since the above mentioned materials are heavy metals which may be cytotoxic to cause cell apoptosis, the usage of these materials in organisms is limited. Researchers then developed non-heavy metals as semiconductor nanomaterials (e.g. ZnSe, InP, InAs, etc); however, the wavelengths of emission light of these materials are in the range of visible spectrum, making these materials difficult to be used in organisms. Wilcoxon et al. developed a 5-nm fluorescence gold nanoparticle by using non-semiconductor materials with less toxicity, e.g. gold, silver . . . etc. It also exhibits quantum confinement effect when manipulating the synthesized size of fluorescence gold nanoparticles to be 2-20 nm, making the materials show discrete electronic state structure which is similar to atoms. Therefore, gold clusters are suitable for the application of bioimaging. Controlling size of gold clusters, using poly(amidoamine) dendrimer, mercaptoundecanoic acid, lipoic acid . . . etc. as ligands, the wavelength of emitted fluorescence can range from visible spectrum to near infrared spectrum. Recently, due to the advancement of green chemistry, one can also use bovine serum albumin (BSA) as template for aqueous phase distribution of gold clusters in order to be easily used in biomedical area. Because that the size of gold nanocluster is extremely small, the gold nanocluster can exhibit discrete electronic state structure similar to atom due to quantum confinement effect. After the gold nanoclusters accept the energy, the electron will jump from highest occupied molecular orbital (HOMO) to lowest unoccupied molecular orbital (LUMO), and then excite the light energy which is called fluorescence.

Osteoarthritis has become one of the influential diseases affecting the elderly. Further, the osteoarthritis continues to worsen during course of disease, finally leading to artificial joints replacement surgery. It's considerably time-consuming and expensive, and usually brings lots of inconveniences in daily life during treatment. Researchers have found some medicines that might change the courses of osteoarthritis. At present time, people use MRI system to observe early osteoarthritis. However, the cost of using MRI system is too high to be affordable for every patient. Thus, the MRI system is used mostly in research purpose. Apart from this, there are no other reliable tools for effective detection of osteoarthritis. Once patients miss the golden opportunity to treat early osteoarthritis, the patients won't feel much different even though they use drugs that will change the course of osteoarthritis, because that the damage to cartilage will have been irreversible. Therefore, there is an urgent need for developing a reliable diagnosis tool for effective detection of osteoarthritis, so that one can detect osteoarthritis as early as possible before the damages to articular cartilages become irreversible, and use medications to change the courses of osteoarthritis to prolong the joints' life, making the osteoarthritis no longer a troubled disease.

### BRIEF DESCRIPTION OF THE DRAWINGS

FIG. 1 is a schematic diagram of the early osteoarthritis detection system (hv indicates the energy of an incident photon).

FIG. 2 shows that changing the amount of silver nitrate (AgNO₃) will produce gold nanorods with different absorption peaks.

FIG. 3 shows that changing the amount of ascorbic acid will produce gold nanorods with different absorption peaks.

FIGS. 4A-4B show the ultraviolet-visible (UV-Vis) absorption spectrums of gold nanorod and modified gold nanorod. FIG. 4A: before process improvement; FIG. 4B: after process improvement.

FIG. 5 shows the analysis results of the surfactant “hexadecyl-trimethyl-ammonium bromide (CTAB)” on the surface of gold nanorod (GNR), and the functional groups of CTAB.

FIG. 6 shows the analysis results of the surface modifier “cysteamine hydrochloride” and the surface functional groups on the gold nanorod (GNR).

FIG. 7 shows the analysis results of surface ligand “lipoic acid” and the surface functional groups on the gold nanocluster.

FIG. 8 shows the result of using fluorescein isothiocyanate (FITC) as a fluorescence probe model to react with different concentrations of gold nanorod (GNR) for different days.

FIG. 9 shows the feasibility of using FITC as a fluorescence probe model in vitro (ck: cathepsin K).

FIG. 10A shows that the line having higher peak is the fluorescence value of 0.005 mM peptide (SEQ ID NO: 1) binding with FITC; the line having lower peak is the fluorescence value of the peptide after binding with GNR (the fluorescence value approaches to 0). This result indicates that almost all peptides have bound to gold nanorod. Hence, 0.005 mM is set as the peptide concentration for reaction. FIG. 10B shows the result of fluorescence light intensity under cleavage by different volume of cathepsin K.

FIG. 11 shows the fluorescence spectrum of gold nanoclusters at different days.

FIG. 12 shows the fluorescence emission spectrum of gold nanocluster (gnc) and the absorption spectrum of gold nanorod (gnr).

FIG. 13 shows the fluorescence spectrum of fluorescence probes that are made of difference amounts of gold nanoclusters.

FIGS. 14A-14C show the transmission electron microscopy (TEM) images of metal nanoparticles. FIGS. 14A and 14B show gold nanorods with different sizes due to variable changes during synthesis; FIG. 14C shows gold clusters with spectrum in near infrared region.

FIG. 15 shows the result of all gold fluorescence probe of the present invention detecting ADAMTS-4 enzyme, wherein the peptide on the probe is SEQ ID NO: 2.

FIG. 16 shows the result of all gold fluorescence probe of the present invention detecting ADAMTS-4 enzyme, wherein the peptide on the probe is SEQ ID NO: 3.

FIG. 17 shows the result of all gold fluorescence probe of the present invention detecting MMP-13 enzyme, wherein the peptide on the probe is SEQ ID NO: 4.

FIG. 18 shows the micro computed tomography (MicroCT) image of gold nanoclusters in vitro. AuNCs indicates gold nanoclusters; DDW indicates double distilled water.

FIG. 19 shows the micro computed tomography (MicroCT) image of gold nanoclusters in vivo.

FIG. 20 shows the GNC calibration curve.

FIG. 21 shows the cathepsin K calibration curve.

FIG. 22 shows the detection result of the all gold fluorescence probe system against cathepsin K from synovial fluid of SD rats with arthritis induced by IL-1β.

FIG. 23 shows the detection result of the all gold fluorescence probe system against cathepsin K from synovial fluid of Nubian goats.

FIG. 24 shows the GNC calibration curve.

FIG. 25 shows the ADAMTS-4 calibration curve.

FIG. 26 shows the detection result of the all gold fluorescence probe system against ADAMTS-4 from synovial fluid of SD rats with arthritis induced by IL-1β.

FIG. 27 shows the detection result of the all gold fluorescence probe system against ADAMTS-4 from synovial fluid of Nubian goats.

FIG. 28 shows the GNC calibration curve.

FIG. 29 shows the MMP-13 calibration curve.

FIG. 30 shows the detection result of the all gold fluorescence probe system against MMP-13 from synovial fluid of SD rats with arthritis induced by IL-1β.

FIG. 31 shows the detection result of the all gold fluorescence probe system against MMP-13 from synovial fluid of Nubian goats.

### SUMMARY OF THE INVENTION

The present invention relates to a gold fluorescence resonance energy transfer nanoprobe comprising a gold fluorescence donor, a gold fluorescence acceptor, and a linker fragment that connects the gold fluorescence donor and the gold fluorescence acceptor, wherein the fluorescence resonance energy transfer is carried out between the gold fluorescence donor and the gold fluorescence acceptor. This all gold probe employing fluorescence resonance energy transfer technique can be used for detecting diseases such as arthritis, osteoporosis, and cancer metastasis.

### DETAILED DESCRIPTION OF THE INVENTION

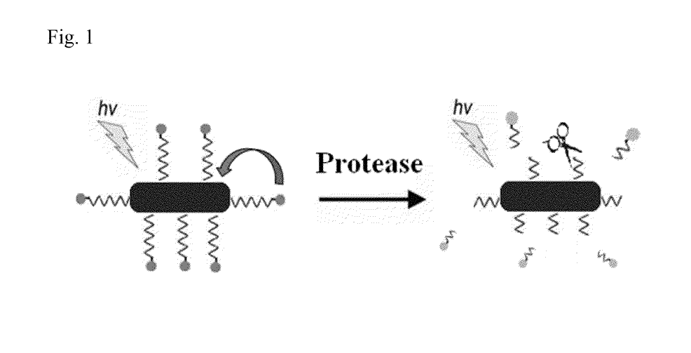

The present invention is based on energy transfer theory to develop a type of fluorescence probe used as a disease (e.g. arthritis, osteoporosis, or cancer metastasis) detecting system. The present invention uses gold nanorod (GNR) as a substrate to be modified with a short peptide chain which can be cleaved by a protease produced by nidus (e.g. cathepsin K produced by osteoarthritis). The end of the short peptide chain is further grafted with a gold nanocluster which can emit light with near infrared wavelength. Take osteoarthritis as an example, the fluorescence probe that is not cleaved by cathepsin K doesn't appear fluorescence due to the energy transfer. On the other hand, once the cathepsin K works, the gold nanocluster will be detached from GNR, resulting in fluorescence emission, which means the existence of osteoarthritis. The schematic diagram of the system is shown in FIG. 1.

Therefore, the present invention provides a gold fluorescence resonance energy transfer nanoprobe comprising a gold fluorescence donor, a gold fluorescence acceptor, and a linker fragment that connects the gold fluorescence donor and the gold fluorescence acceptor, wherein the fluorescence resonance energy transfer can be carried out between the gold fluorescence donor and the gold fluorescence acceptor. In an embodiment, the linker fragment is a peptide with a length of 3-20 amino acids, and the peptide can be cleaved by an enzyme so that the fluorescence resonance energy transfer between the gold fluorescence donor and the gold fluorescence acceptor no longer occurs, wherein the activity or concentration of the enzyme in a patient or a nidus (e.g. arthritis site) is higher than in a normal subject or normal tissue. The enzyme includes but not limit to matrix metalloproteinase (e.g. MMP-13), cathepsin (e.g. cathepsin K), or aggrecanase (e.g. ADAMTS-4). In an embodiment, the gold fluorescence acceptor is a gold nanorod with a length of 50-200 nm, and an absorption wavelength of 470˜570 nm and/or 600˜950 nm. In an embodiment, the gold fluorescence donor is a fluorescence gold nanocluster with a length of 5-50 nm, and an emission wavelength of 600˜950 nm. In an embodiment, the gold fluorescence resonance energy transfer nanoprobe can be used for in vivo or in vitro detection. Furthermore, the gold fluorescence resonance energy transfer nanoprobe of the present invention can also be used in X-ray contrast imaging.

### EXAMPLES

The examples below are non-limiting and are merely representative of various aspects and features of the present invention.

### Example 1

### Materials and Methods:

### Synthesis of Gold Nanorods (GNRs)

Gold nanorods were synthesized by a seed-mediated growth method comprising a mixing seed solution and a growth solution. The amount of AgNO₃ and ascorbic acid in growth solution would affect the synthesis of the gold nanorods in length and shape. After the completion of synthesis, an ultraviolet-visible (UV-Vis) absorption spectrometer was used for preliminary identification of the gold nanorods.

I. Seed Solution
A magnetic stir bar was put into a flask, and then hexadecyl-trimethyl-ammonium bromide (CTAB) (0.2 M, 3.75 ml) and HAuCl₄.3H₂O (0.0005 M, 5 ml) were added. The solution was stirred until becoming yellow, then NaBH₄ (0.1M, 0.6 ml) was quickly added, resulting in the formation of a brownish yellow solution. The solution was kept 2 hours for reaction.

II. Growth Solution
A magnetic stir bar was put into a flask, then CTAB (0.2 M, 15-17 ml), HAuCl₄.3H₂O (0.001 M, 15 ml), and AgNO₃ (0.0040 M, 0.625 ml) were add (the amount of AgNO₃ was the main factor affecting GNR length). The solution was stirred until becoming yellowish, and then ascorbic acid (0.0788 M, 325 ml) was added to obtain a colorless solution.

III. Final Step
An appropriate amount of the seed solution (420 μl) was added into the growth solution and stirred. After reaction for 12 hours, the GNR solution was obtained and the color of the solution was from blue to dark red due to the amount of AgNO₃. The solution was centrifugated to remove excess CTAB and collect solid GNRs. Double distilled water (DDW) was added to re-suspend the sedimentary GNRs. This step (centrifugation and re-suspension) was repeated for 1 to 3 times to obtain stable GNRs.

### GNR Surface Modification

Because the protecting group “CTAB” on the GNR surface was positively charged, it was cytotoxic and would cause cell apoptosis after entering organisms. Thus, the GNR surface should be modified to bind to other molecules easily. The surface modifier used was cysteamine hydrochloride, which had two functional groups, one end was a SH group that could bind to GNRs, and another end was an amino group that could bind to other molecules. The cysteamine hydrochloride was used to modify the GNR surface to make exposed amino groups on the GNR surface.

GNRs (1 ml) and cysteamine hydrochloride (100 μl, 25 mM) were mixed together, then an appropriate amount of MeOH was added to detach the CTAB molecules from GNRs. The solution was shocked and heated. After the temperature gradually raised to 50° C., the solution was kept for several hours for reaction, and placed for cool down. The solution was centrifugated (7000-7300 rpm, 15° C.) to remove excess CTAB molecules and cysteamine hydrochloride and collect modified GNRs. Double distilled water was added to re-suspend the modified GNRs. This step (centrifugation and re-suspension) was repeated twice.

Binding of Peptides that could be Cleaved by Cathepsin K

Utilizing amidization reaction, the peptide (0.005 mM) (QCGKPG, SEQ ID NO: 1), which was bonded with fluorescein isothiocyanate (FITC) and cleavable by cathepsin K, and 1-Ethyl-3-(3-dimethylaminopropyl)carbodiimide (EDC) were mixed by the equivalent (eq) of 1:1.5. K₂CO₃ (5 mM), acting as a catalyzer, was added to provide a base environment to activate the carboxyl group on the peptide. Purified GNRs with amino groups exposed on the surface was then added and reacted at 4° C. The compounds not bonded to GNRs were removed by centrifugation.

### The Cleavage Effect of Cathepsin K

The GNRs obtained from centrifugation was bonded to the peptide product. A solution of cathepsin K (2 μg, 80 μl) was prepared and then diluted to 1 ml. 5-100 μl of the diluted cathepsin K were added to GNRs and kept for 24 hours for reaction. The solution was centrifugated and the supernatant was collected. The fluorescence intensity of free FITC on the peptide cleaved by cathepsin K was determined.

### Water-Soluble Fluorescence Gold Nanoclusters

The fluorescence probe to be developed was expected to be useful in detection in vivo, hence the light-emitting substances used for detection should be able to pass through living body without being absorbed by tissues or causing tissue damage. Therefore, the reasons for using gold nanoclusters as light-emitting groups were not only the emitted light wavelength was in the near infrared region so that gold nanoclusters could work with GNRs to form a probe by FRET theory, but also the gold nanoclusters were made by gold material which has better bio-compatibility compared with other light-emitting groups.

The synthesis method of gold nanoclusters was described below. The decyclization was carried out by reaction of 3 equivalents of lipoic acid and 0.1 M NaOH for 15 minutes. Then 1 equivalent of aqueous chlorauric acid was added to react for several minutes. Two equivalents of NaBH₄ solution were added for reaction for tens of minutes to reduce gold. An appropriate amount of MeOH was added and then the solution was drained. This step was repeated, and then double distilled water was added to evenly disperse the gold nanoclusters. The solution was centrifugated by using 10 kDa molecular sieve tube (nanofilter) at 7500 rpm for 15 minutes, and then rinsed with double distilled water. This step was repeated twice to remove unreacted substances and excessively small gold nanoclusters. The gold nanoclusters was stocked in a solution with pH around 9.

### All Gold Fluorescence Probe System

An all gold fluorescence probe system was synthesized by imitating FITC fluorescence probe system. The modified GNR was used as a substrate. The peptides cleavable by cathepsin K and gold nanoclusters of which the emitting light wavelength located in near infrared region were gradually bonded to GNR by amidization reaction. Finally, an all gold fluorescence probe system, which could be used as a detection tool for early osteoarthritis, was formed.

Peptide-2 (SEQ ID NO: 1) had two functional groups, one was carboxyl group (—COOH), and another was amino group (—NH₂). The amino group had to be pre-protected to prevent the bindings between peptides when carboxyl group was activated. 1.2 equivalents of fluorenylmethyloxycarbonyl chloride were added into 1 equivalent of peptide-2 solution for reaction of 6 hours to protect amino groups. 1.2 equivalents of 1-Ethyl-3-(3-dimethylaminopropyl)carbodiimide (EDC) solution was then added for reaction of 2-4 hours to activate carboxyl groups. GNRs modified with amino groups (collected after centrifugation) were added for reaction of 1.5 days. The solution was centrifugated (7000-7300 rpm, 15° C., 25 minutes) to collect the product, and then the double distilled water was added to re-suspend the product. This step was repeated twice to obtain the GNRs on which peptide-2 was bonded.

20% piperidine in dimethyl formamide (DMF) was added into aforementioned GNR product bonded with peptide-2 having fluorenylmethyloxycarbonyl chloride protecting group to suspend GNRs in DMF solution for reaction of about 4 hours. The solution was centrifugated at 7000-7300 rpm for 25 minutes to collect purified deprotected GNRs.

An appropriate amount of 1-Ethyl-3-(3-dimethylaminopropyl)carbodiimide (EDC) was added into gold nanocluster solution for reaction of 2-4 hours. Then 0.1 ml DMF and aforementioned GNRs on which the bonded peptide-2 was deprotected to expose its amino group were added for reaction of 1.5-3 days to make bonding between the gold nanoclusters and the peptide-2 on GNRs. The solution was centrifugated at 7000-7300 rpm for 25 minutes to collect the all gold fluorescence probes.

### Detection by all Gold Fluorescence Probes In Vitro

5˜100 μl of cathepsin K solution (2 μg, 80 μl) pre-activated in weak acid was added into the all gold fluorescence probes after centrifugation for reaction of 24 hours. The solution was centrifugated to collect the supernatant, and then the fluorescence of it was determined to obtain in vitro detection result of the all gold fluorescence probe system.

### Results:

### Variables in GNR Synthesis Experiment

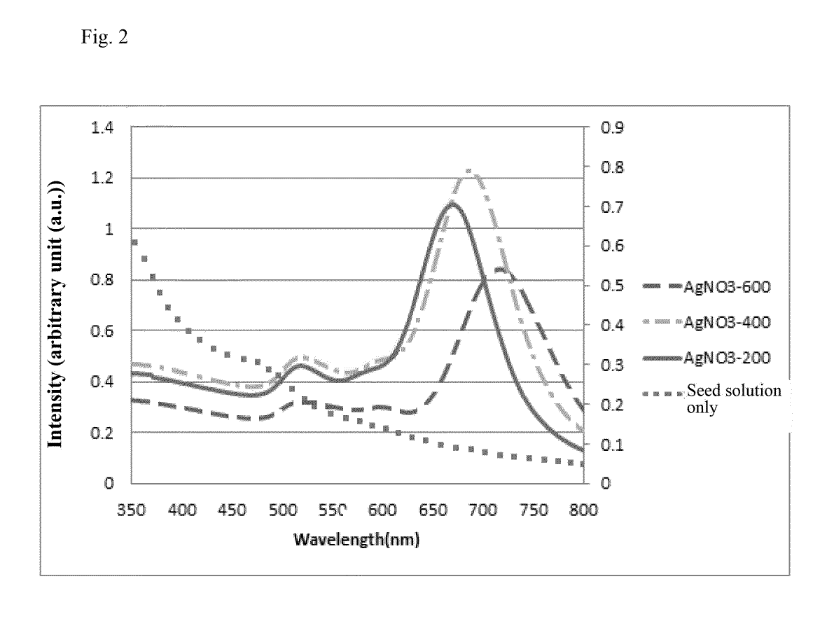

One of the important variables in GNR synthesis was the concentration of AgNO₃ as it affected the absorption peak of GNR. While the addition amount of AgNO₃ was at its maximum of 600 ml, the absorption peak was red-shifted with a maximum absorption wavelength of 728 nm. In contrast, when the addition amount of AgNO₃ was at its minimum of 200 ml, the absorption peak was blue-shifted with a maximum absorption wavelength of 658 nm (FIG. 2).

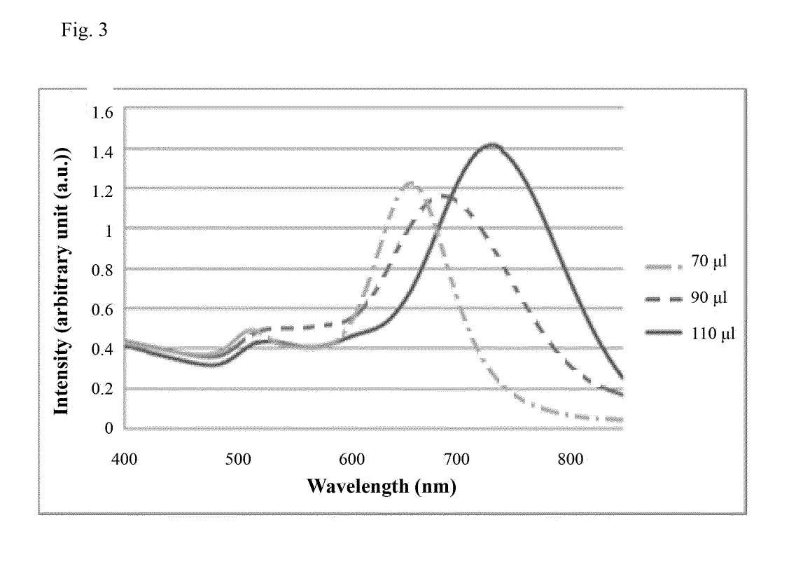

During GNR synthesis, the absorption peak exhibited a redshift in the presence of a small quantity of acid (e.g. hydrochloric acid, nitric acid). Since the ascorbic acid used in the example was an acidic solution, the shift phenomenon existed as well as different amounts of ascorbic acid were added. While the addition amount of ascorbic acid was at its maximum of 110 μl, the absorption peak had a redshift with maximum absorption wavelength of 734 nm; however, while the addition amount of ascorbic acid was at its minimum of 70 μl, the absorption peak had a blueshift with maximum absorption wavelength of 668 nm (FIG. 3).

### Surface Modification of GNRs

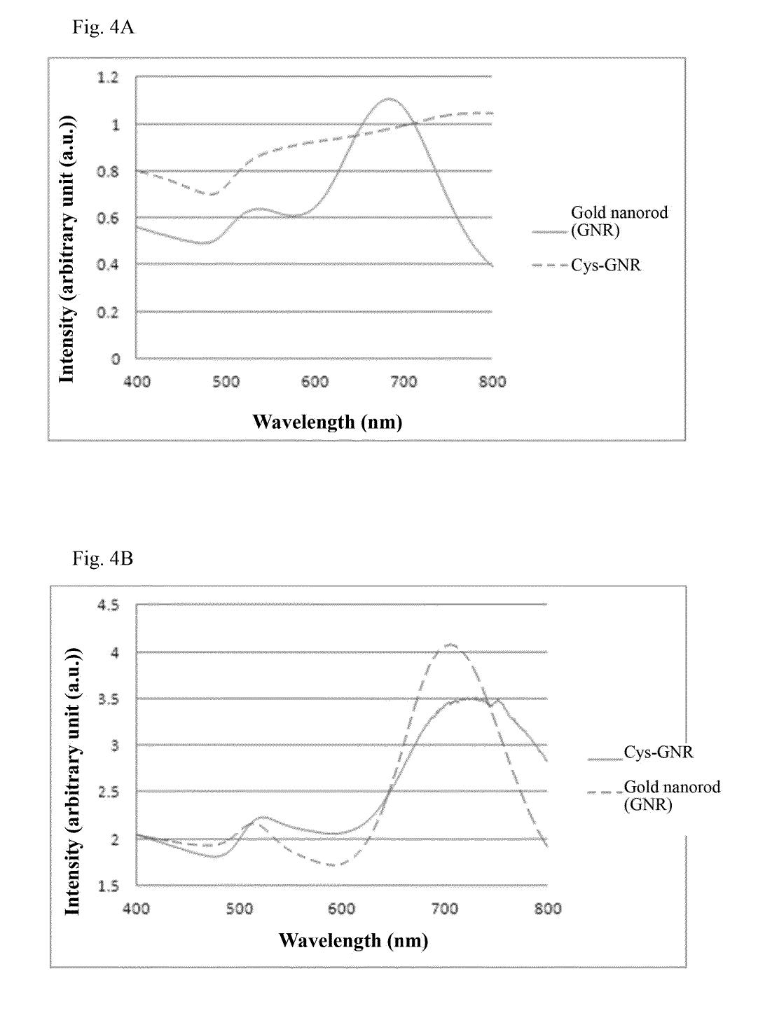

During GNR surface modification, CTAB detachment from GNR surface was required by heating in order to make bonding between cysteamine hydrochloride and GNRs available. As shown in FIG. 4A, when the temperature rose quickly, the cysteamine hydrochloride couldn't bond to the GNR surface well, causing the aggregate of GNRs and the change of absorption peak. However, when the temperature was under control to rise slowly, the original absorption peak of GNR did not change too much so that the optical properties wouldn't be affected (FIG. 4B).

### Infrared Spectroscopy

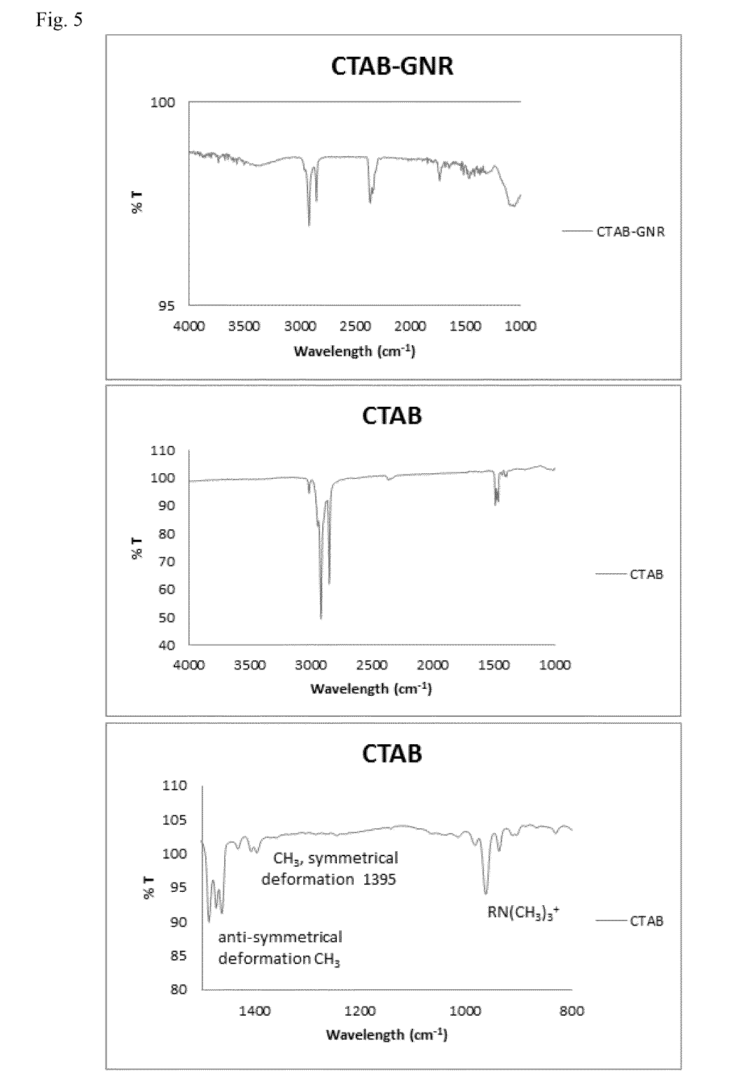

CTAB-GNR Infrared Spectroscopy:
The surfactant, CTAB, was a quaternary alkyl amine with long carbon chain. CTAB did not contain obvious functional groups. The C—H bond in CTAB could vibrate in symmetric and antisymmetric stretching at 2856 cm⁻¹ and 2919 cm⁻¹ respectively; the CH₃ exhibited symmetric and antisymmetric deformation vibration at 1398 cm⁻¹ and 1463 cm⁻¹ respectively; and the quaternary amine C—N⁺ could exhibit stretching vibration at 962 cm⁻¹. As shown in FIG. 5, the GNR also exhibited vibration signals at similar wavelength.

### Cysteamine-GNR Infrared Spectroscopy:

The GNR surface was modified with cysteamine to replace the original protecting group CTAB, and infrared spectroscopy was used to determine if the cysteamine really bond to the GNR surface. Because that cysteamine was a highly absorbent compound, the peak width of its amino group was wider. Cysteamine was a primary amine compound that the N—H bond exhibited bending and stretching vibration at 1603 cm⁻¹ and 1281 cm⁻¹ respectively, and S—H bond generated signal at 2492 cm⁻¹.

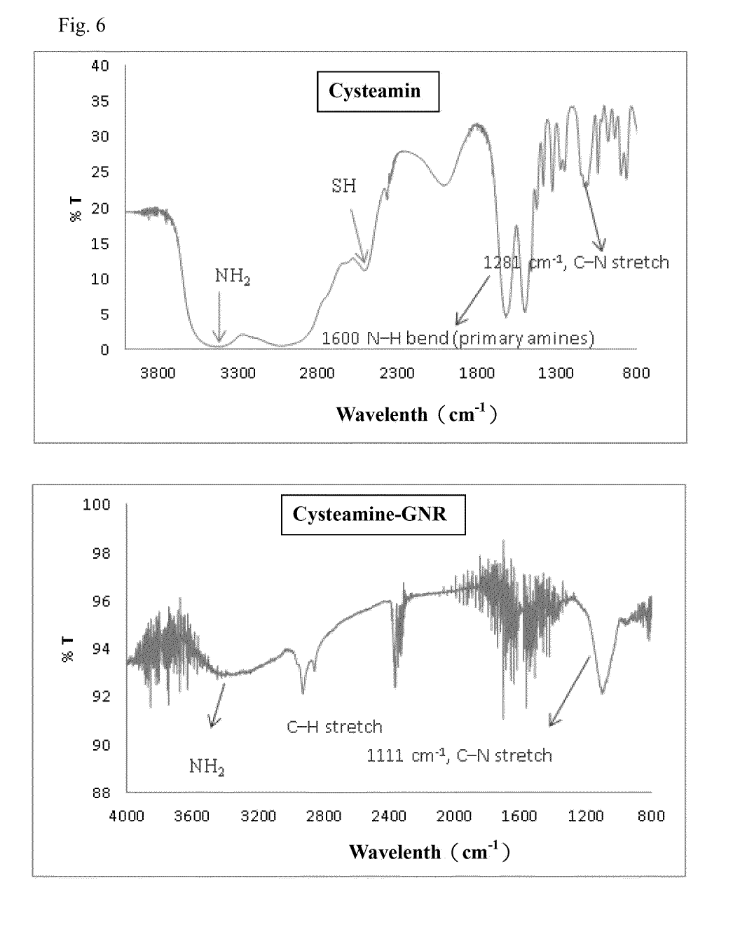

The signal wavelengths of cysteamine-GNR were generally the same as the wavelengths of cysteamine. However, since the sulfur of cysteamine was bonded to gold, the S—H signal of cysteamine at 2492 cm⁻¹ couldn't be observed in cysteamine-GNR infrared spectrum. Moreover, the signal of CTAB at 962 cm⁻¹ couldn't be observed in cysteamine-GNR infrared spectrum either, indicating that the GNR surface was successfully modified with cysteamine (FIG. 6).

### Gold Nanocluster Infrared Spectroscopy:

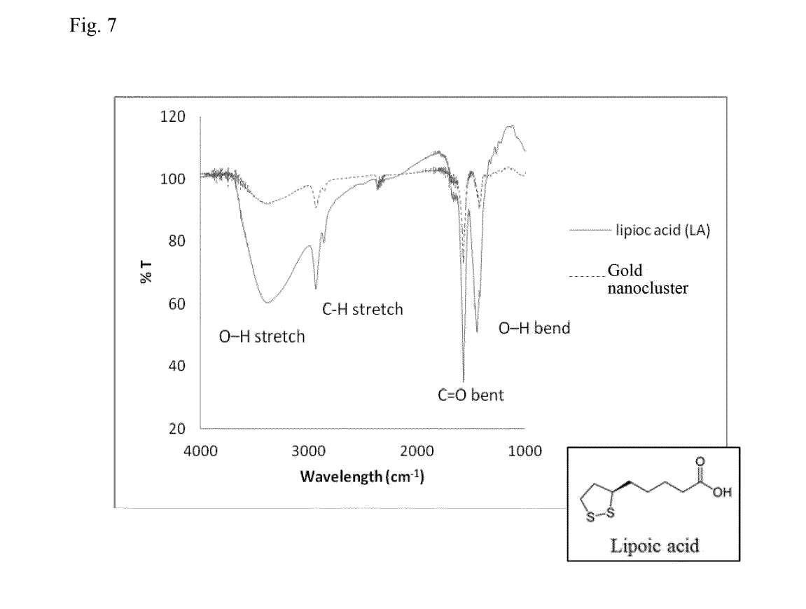

The lipoic acid was an outside ligand of gold nanoclusters, acting as a major identification molecule. One could identify the functional groups on lipoic acid by infrared spectroscopy. The C—H bond of lipoic acid could vibrate in symmetric and antisymmetric stretching at 2940 cm⁻¹ and 2896 cm⁻¹ respectively; the C═O bending vibration was at 1567 cm⁻¹, the O—H stretching vibration was at 3400 cm⁻¹, and the O—H bending vibration was at 1448 cm⁻¹. The gold nanoclusters synthesized by lipoic acid also generated similar signals at corresponding wavelengths (FIG. 7).

### Photoluminescence Spectroscopy

The fluorescence would quench due to the energy transfer effect. The photoluminescence spectroscopy was used to determine the fluorescence quench effect and the feasibility of enzyme cleavage in vitro.

Using FITC as a Fluorescence Probe Model:

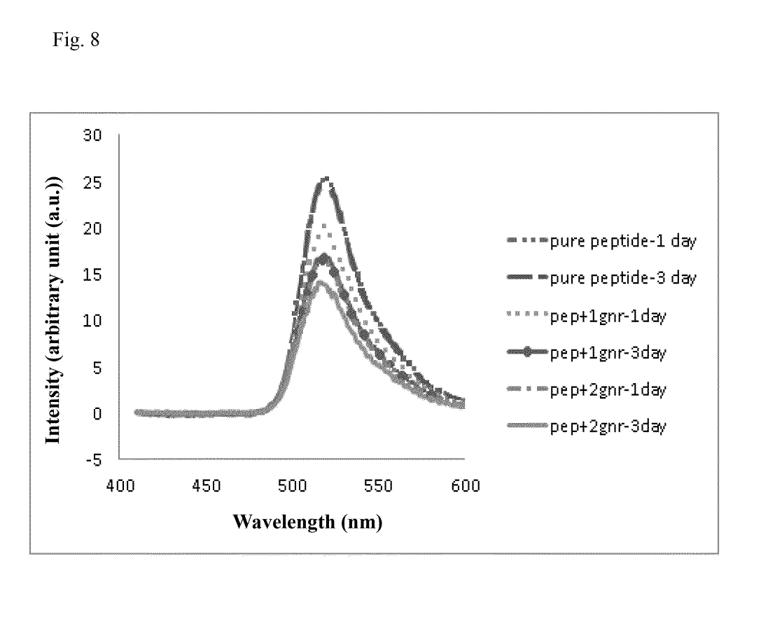

The FITC was used as primary fluorescence probe. As shown in FIG. 8, the fluorescence intensities of the peptide bonded with fluorescent substance maintained the same at day 1 and day 3, implying that FITC would not abate in short time. Therefore, one could regard it as a standard. Since the reaction mainly occurred in PBS, when the GNR concentration exceeded twice as the original GNR concentration, GNR tended to aggregate, resulting in poor reaction. Therefore, the present invention merely compared the fluorescence intensities of 1-fold and 2-fold of GNR concentrations. As shown in FIG. 8, the most obvious quench effect was observed after 3 days of reaction of 2-fold GNR concentration and FITC fluorescence peptide.

The Feasibility of FITC Fluorescence Probe In Vitro:

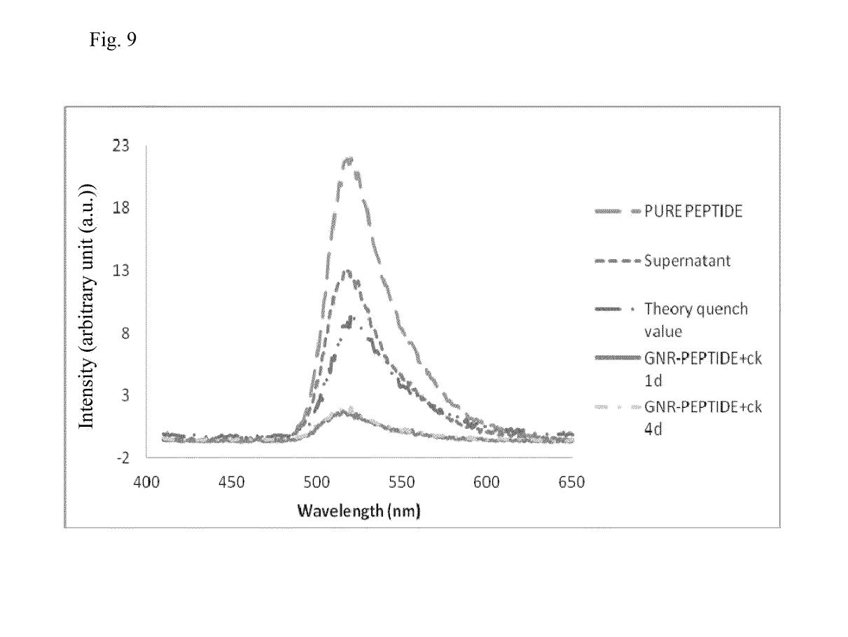

The group of 2-fold GNR concentration with 3-day reaction time as described above was used for following experiments. As shown in FIG. 9, after reaction and centrifugation, the supernatant was regarded as unbound peptide and the fluorescence of the supernatant was determined (shown as the line with second highest peak in the figure, marked as “supernatant”). The fluorescence value quenched in the experiment (shown as the line with third highest peak, marked as “theory quench value”) was obtained by subtracting the supernatant fluorescence from the original peptide fluorescence (shown as the line with highest peak in the figure, marked as “pure peptide”). The quenched fluorescence value was 10 OD. The cathepsin K (7.69×10⁻⁸ M, 200 μl) was then added into the peptide-GNR solution for 1 day of reaction to release fluorescent substance, and then the fluorescence was determined. The percentages of quenched fluorescence and released fluorescence were calculated by a software. The percentage of quenched fluorescence was 42.11%, while the percentage of released fluorescence after cleavage by cathepsin K was 20.83%.

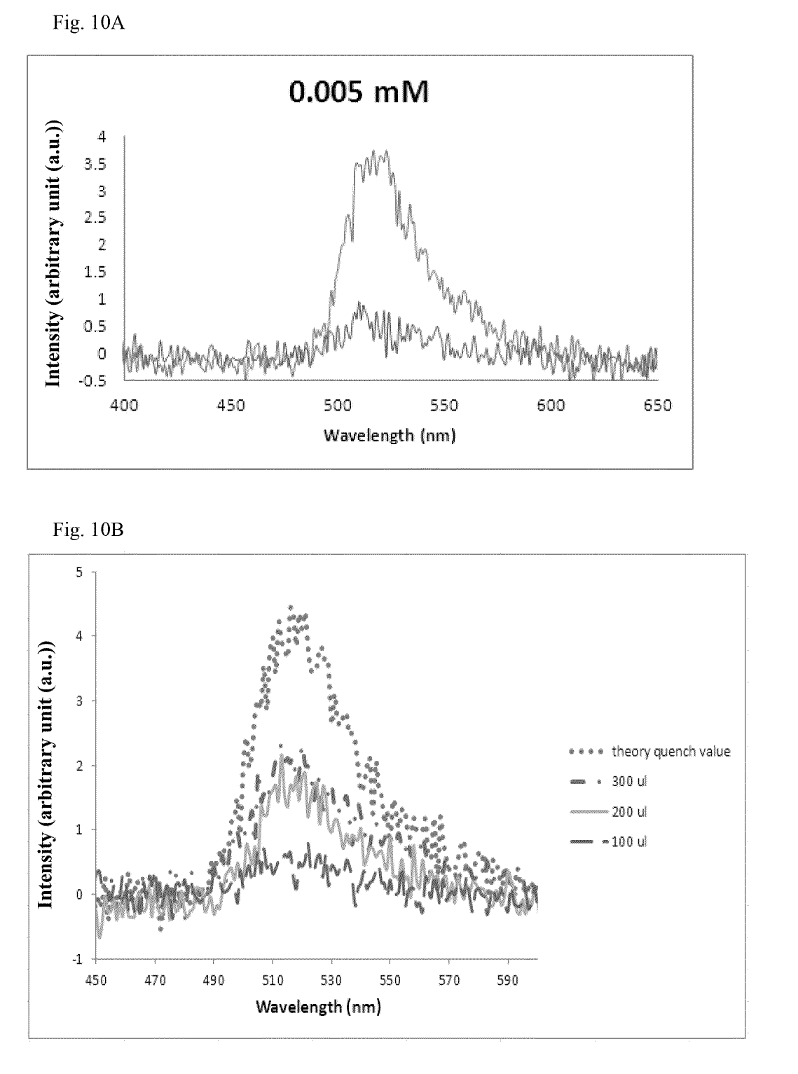

Since about half amount of peptide added as aforementioned was not bonded to the GNR surface, the peptide concentration was lowered to make the fluorescence value of supernatant (the solution containing unbound peptide) approach zero, and then cathepsin K was added for investigation. As shown in FIG. 10, the line with the higher peak in FIG. 10A was the fluorescence of 0.005 mM peptide (SEQ ID NO: 1) bonded with FITC. The fluorescence value approached zero after the peptide bonded to GNRs (the line with the lower peak), indicating that the peptide had completely bonded to GNRs. Therefore, 0.005 mM was set as the peptide concentration for being added in the reaction. Three cathepsin K solutions were prepared in same concentration with different volume of 100 μl, 200 μl, and 300 μl. After reaction with peptide-GNR solution for 1 day, the solutions were centrifuged to collect the supernatant. The fluorescence of the supernatant was determined and shown in FIG. 10B.

Gold Nanoclusters:

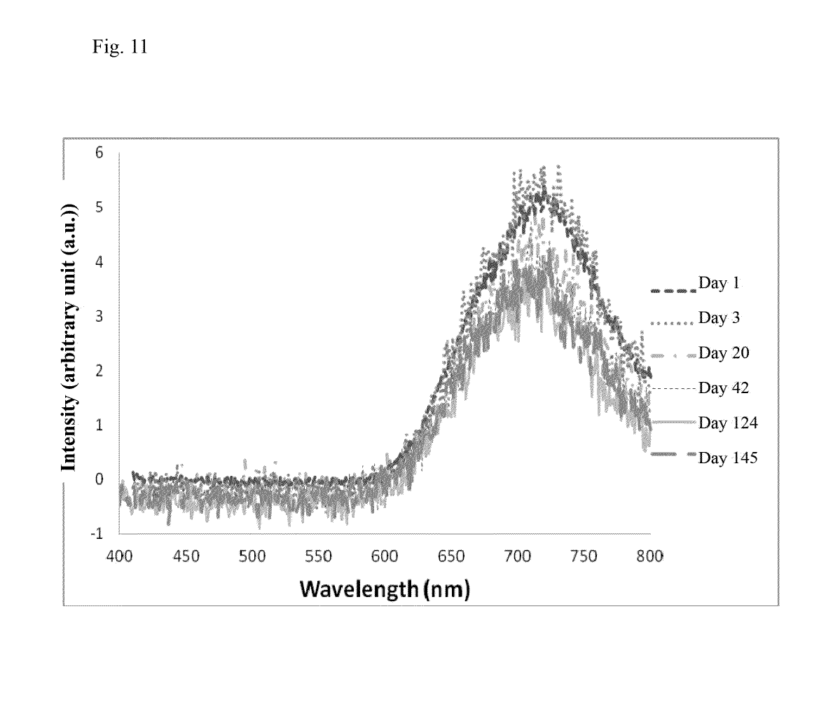

By the ratio of chloroauric acid: lipoic acid=1:3, gold nanoclusters were synthesized with the ability of emitting red light in near infrared region, and the emission wavelength was between 710-730 nm. Furthermore, the gold nanoclusters could be kept in sodium bicarbonate with a pH of about 9 for a lone time, at least 3 months. However, the decay of fluorescent substance as well as gold nanocluster was yet unavoidable, but the decay degree was not too obvious (FIG. 11).

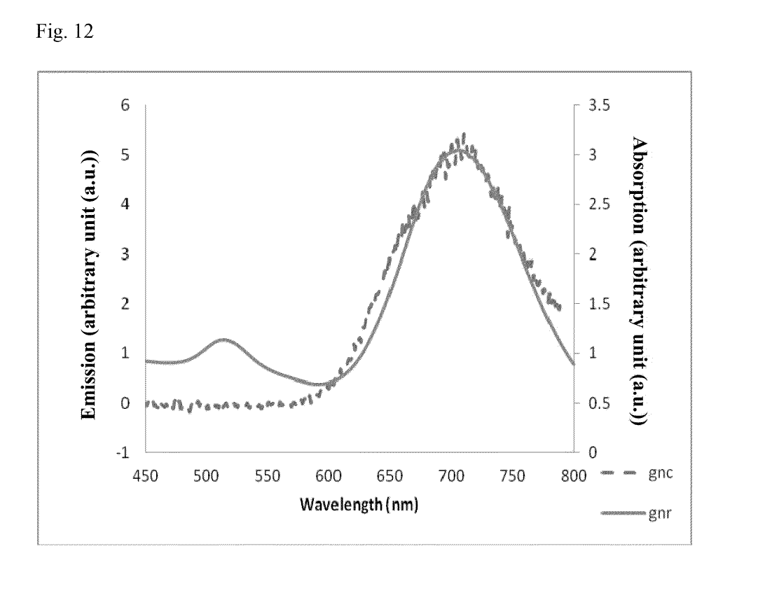

The FRET system was utilized to design a gold nanocluster fluorescence probe, while it was based on a premise that the energy transfer between the energy donor “gold nanocluster (GNC)” and the energy acceptor “gold nanorod (GNR)” was complete. As shown in FIG. 12, the emission spectrum of GNCs completely overlapped with the absorption spectrum of GNRs. Therefore, once the distance at which the energy transfer was efficient between GNC and GNR, the fluorescence quenched. In contrast, once the cathepsin K was present to cleave the peptide, making the GNC leave beyond the efficient distance, the fluorescence appeared again. Therefore, the fluorescence probe system was established.

The Fluorescence Probe Designed by Using GNC:

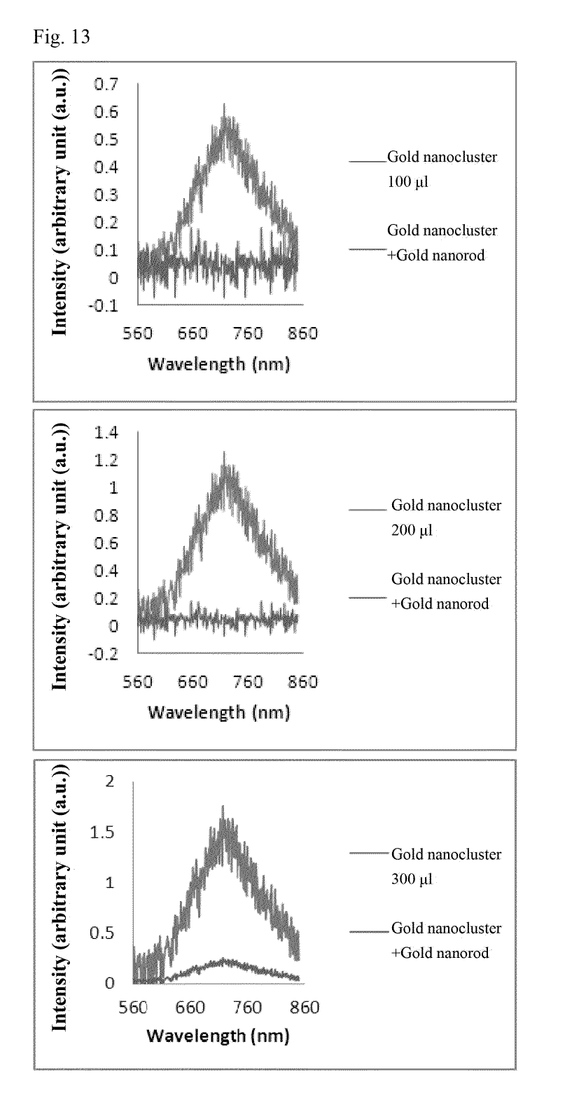

FITC system was used as a reference. Two-fold GNR concentration was used for reaction. GNC solutions with the concentration of 3.25×10⁻⁴ g/ml were prepared and divided into three groups according to different volumes: 100 μl, 200 μl, and 300 μl. After reaction of 3 days, GNCs that did not bond to GNRs were separated by centrifugation. The results were shown in FIG. 13.

### Transmission Electron Microscopy (TEM)

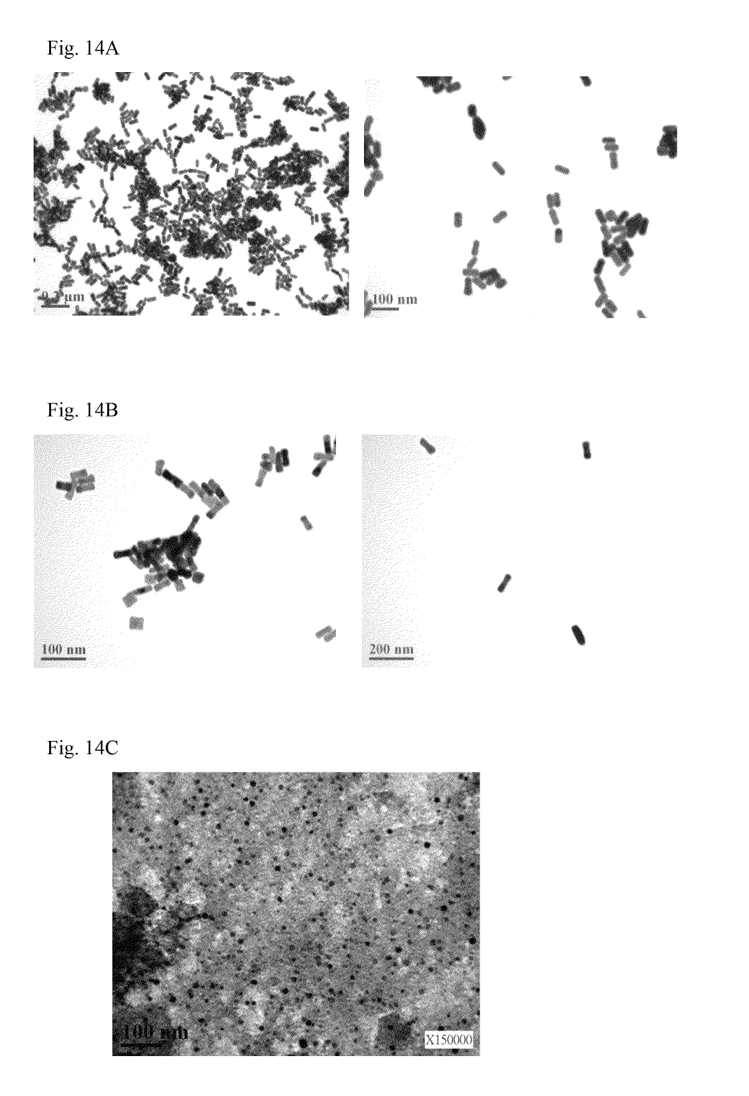

TEM was used to observe the shape of gold nanoparticles (FIG. 14). FIGS. 14A and 14B showed the GNRs with different sizes, such differences were caused by adjusting the variables during synthsis. FIG. 14C showed GNCs of which the emitted light was in near infrared region.

### Example 2

### ADAMTS-4 Cleavage

This example was to discuss the detection result of all gold fluorescence probe of the present invention against ADAMTS-4. The experimental principle was basically the same as described in Example 1. Two peptides called “6mer” (KCEFVG, SEQ ID NO: 2) and “10mer” (GVQEFRGVTG, SEQ ID NO: 3), which were cleavable by ADAMTS-4 but had different lengths, were designed for investigating the relationship between peptide length and cleavage efficiency. Both peptides had two functional groups, one was carboxyl group (—COOH), and another was amino group (—NH₂). The amino group had to be pre-protected to prevent bindings between peptides when carboxyl group was activated. 1.2 equivalents of fluorenylmethyloxycarbonyl chloride were added into 1 equivalent of peptide solution to protect the amino group of peptide. After reaction of 6 hours, 1.2 equivalents of N,N′-dicyclohexylcarbodiimide (DCC) solution were added to activate the carboxyl group. After reaction of 6 hours, GNRs modified with amino groups after centrifugation were added into the solution. After reaction of 1.5 days, the solution was centrifugated to collect the GNR product bonded with peptides. 20% piperidine in dimethyl formamide (DMF) was added into the product for reaction of 4 hours to remove the protective group fluorenylmethyloxycarbonyl chloride. The solution was then centrifugated to collect and purify deprotected GNRs bonded with peptides.

An appropriate amount of 1-Ethyl-3-(3-dimethylaminopropyl)carbodiimide (EDC) was added into the gold nanocluster solution. After reaction of 4 hours, 0.1 ml DMF and aforementioned deprotected GNRs bonded with peptides (on which the amino groups were exposed) were added. After reaction of 1.5 days, the gold nanoclusters bonded to the peptides on GNRs, forming all gold fluorescence probes. The all gold fluorescence probes were then collected by centrifugation.

The all gold fluorescence probes were added into 10 μl of ADAMTS-4 solution (4 μg, 80 μl) which was pre-activated in a weak base. After reaction of 72 hours, the solution was centrifugated to collect the supernatant, and then the fluorescence of supernatant was determined to obtain detection result of the all gold fluorescence probe system in vitro.

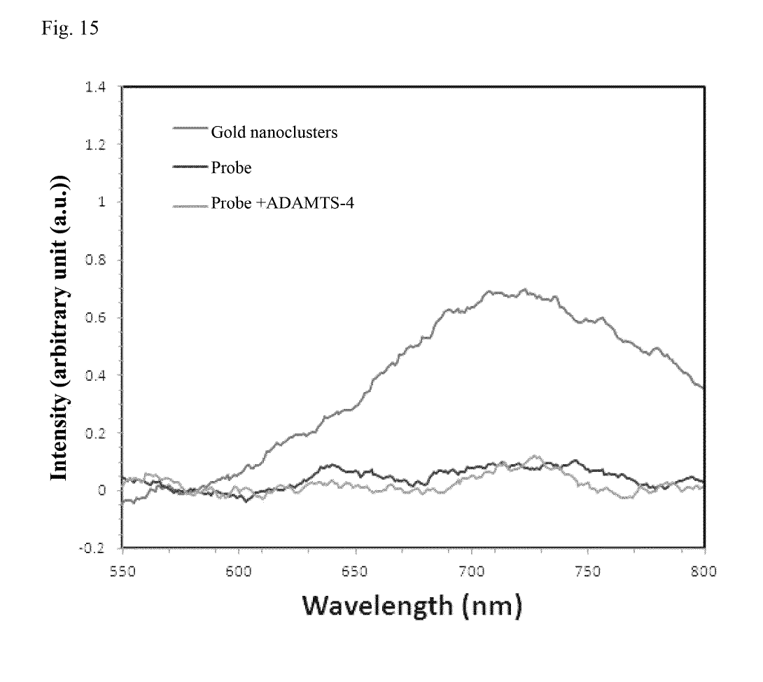

The result of 6mer peptide was shown in FIG. 15. The line marked as “Gold nanoclusters” was the fluorescence of original GNCs; the line marked as “probe” was the quenched fluorescence resulted by binding between GNRs and GNCs; and the line marked as “probe+ADAMTS-4” was the fluorescence after addition of ADAMTS-4 (10 μl, 1×10⁻⁶ M). The result showed that no cleavage occurred (the fluorescence intensity after addition of ADAMTS-4 did not significantly increase compared with that before addition of ADAMTS-4).

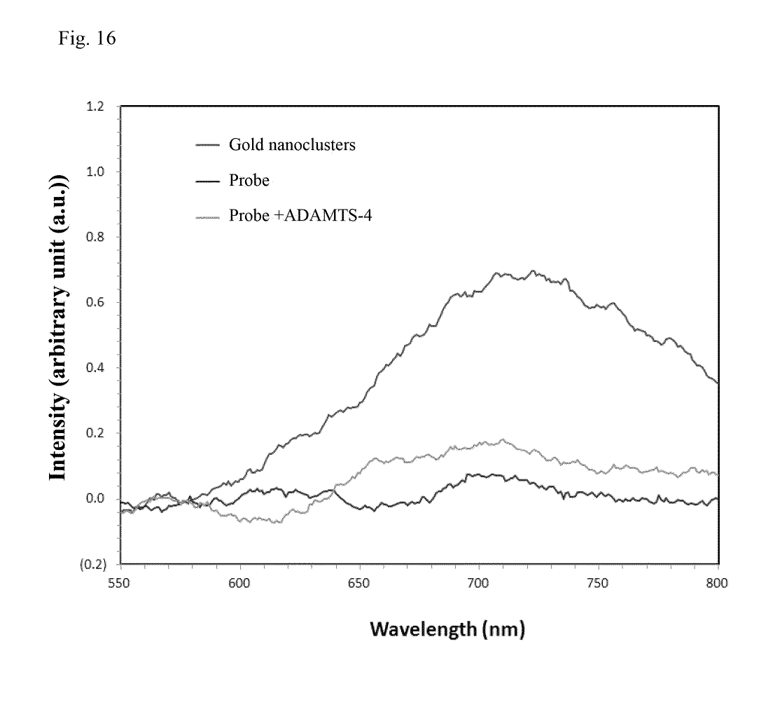

The result of 10mer peptide was shown in FIG. 16. The line marked as “Gold nanoclusters” was the fluorescence of original GNCs; the line marked as “probe” was the quenched fluorescence resulted by binding between GNRs and GNCs; and the line marked as “probe+ADAMTS-4” was the fluorescence after addition of ADAMTS-4 (10 μl, 1×10⁻⁶ M). The result showed that fluorescence was detected, demonstrating that cleavage occurred with a cleavage efficiency of 24%. It was believed that because the molecular weight of ADAMTS-4 was 53 kDa, a longer peptide would have enough space for cleavage by ADAMTS-4 while a shorter peptide was too narrow to be cleaved by ADAMTS-4. Therefore, the cleavage ability of ADAMTS-4 was shown on a longer peptide.

### Example 3

### MMP-13 Cleavage

This example was to discuss the detection result of all gold fluorescence probe of the present invention against MMP-13. The experimental principle was basically the same as described in Example 1. The peptide GPLGVRGKGG (SEQ ID NO: 4) cleavable by MMP-13 was designed. This peptide had two functional groups, one was carboxyl group (—COOH), and another was amino group (—NH₂). The amino group had to be pre-protected to prevent bindings between peptides when carboxyl group was activated. 1.2 equivalents of fluorenylmethyloxycarbonyl chloride were added into 1 equivalent of peptide solution to protect the amino group of peptide. After reaction of 6 hours, 1.2 equivalents of N,N′-dicyclohexylcarbodiimide (DCC) solution were added to activate the carboxyl group. After reaction of 6 hours, GNRs modified with amino groups after centrifugation were added into the solution. After reaction of 1.5 days, the solution was centrifugated to collect the GNR product bonded with peptides. 20% piperidine in dimethyl formamide (DMF) was added into the product for reaction of 4 hours to remove the protective group fluorenylmethyloxycarbonyl chloride. The solution was then centrifugated to collect and purify deprotected GNRs bonded with peptides.

An appropriate amount of 1-Ethyl-3-(3-dimethylaminopropyl)carbodiimide (EDC) was added into the gold nanocluster solution. After reaction of 4 hours, 0.1 ml DMF and aforementioned deprotected GNRs bonded with peptides (on which the amino groups were exposed) were added. After reaction of 1.5 days, the gold nanoclusters bonded to the peptides on GNRs, forming all gold fluorescence probes. The all gold fluorescence probes were then collected by centrifugation.

The all gold fluorescence probes were added into 10 μl of MMP-13 solution (2 μg, 80 μl). After reaction of 72 hours, the solution was centrifugated to collect the supernatant, and then the fluorescence of supernatant was determined to obtain detection result of the all gold fluorescence probe system in vitro.

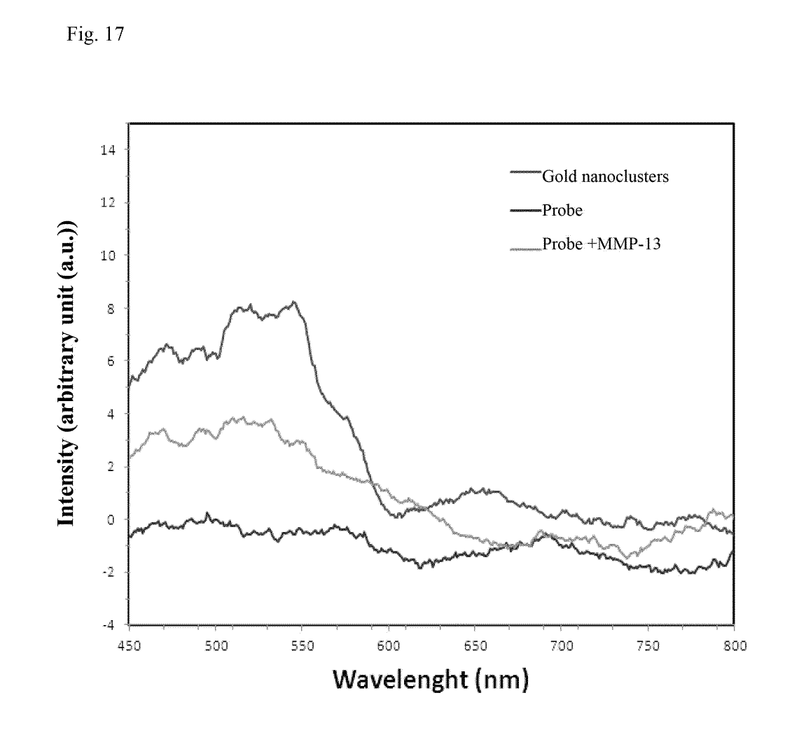

The result was shown in FIG. 17. The line marked as “Gold nanoclusters” was the fluorescence of original GNCs; the line marked as “probe” was the quenched fluorescence resulted by binding between GNRs and GNCs; and the line marked as “probe+MMP-13” was the fluorescence after addition of MMP-13 (10 μl, 4.8×10⁻⁷M). The result showed that the cleavage efficiency of MMP-13 was 52%.

### Example 4

### Computed Tomography (CT) Imaging

Since the X-ray absorption of nanogold was greater than that of traditional iodinated contrast, the lower X-ray dose was able to be applied.

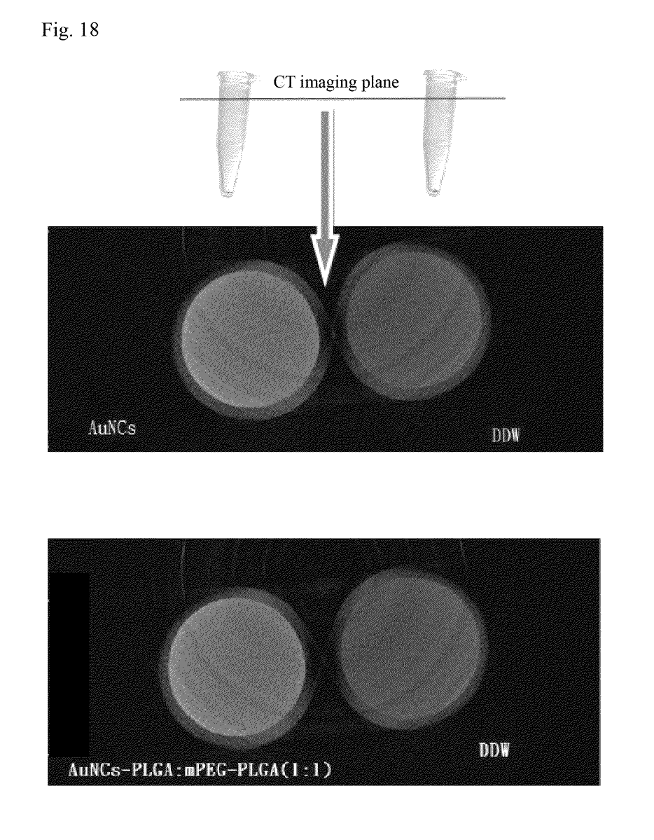

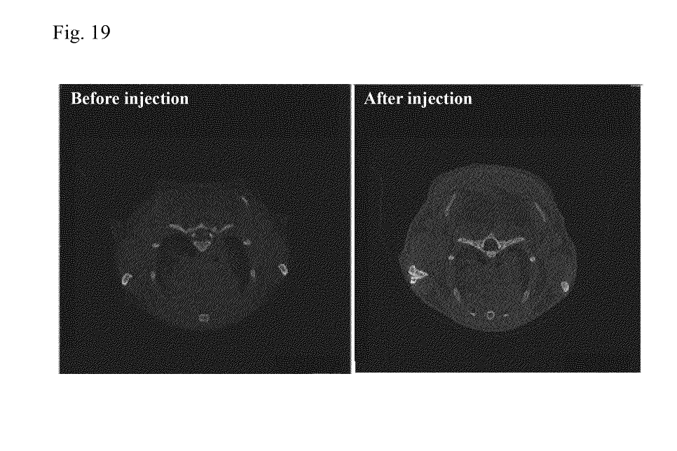

GNCs and double distilled water (DDW) were added into 0.6 ml microtube, and the tube was placed in a micro-CT for animal experiments. As shown in FIG. 18, from the grayscale images of GNCs and DDW, it was observed that the grayscale image of GNCs had an obviously higher grayscale value. Therefore, it was proved that GNCs were contributive to CT imaging. BALB/c strain mice were used for observing the differences between GNC nanoparticles and pure physiological buffer (PBS) by a micro-CT for animal experiments (140 keV, 250 mA, 256-slices with slice thickness of 0.67 mm) in vivo. The in vivo micro-CT imaging was shown in FIG. 19. It was observed that there were grayscale differences before and after injection of GNCs, indicating that GNCs contributed to CT imaging. The all gold fluorescence resonance energy transfer system might be applied in detection of systemic circulation disease by fluorescence or CT imaging in the future.

### Example 5

Non-Invasive In Vivo Imaging System (Caliper IVIS System, IVIS) for Assessment and Measurement of the Detection of all Gold Fluorescence Probe of the Present Invention against Cathepsin K

The non-invasive in vivo imaging system (IVIS) was an imaging system operated with non-radiation to observe experimental animals in vivo. Charge-coupled device (CCD) used in the system was more sensitive than general bioluminescence instrument. The system with higher sensitivity could be used not only in vivo but also in vitro and cell culture experiments. The IVIS system was equipped with specifically-designed counting tray and analysis mode. IVIS was used in this example to investigate the detection result of the all gold fluorescence probe of the present invention against cathepsin K, and then the calibration curve and biological sample result were obtained. Because that fluorescence images displayed in IVIS were contrast values, the differences between samples would affect the results. Therefore, it would be more precise to place GNC sample (for making GNC calibration curve), cathepsin K sample (for making cathepsin K calibration curve) and biological sample in the same 96-well plate for assessment.

### Assessment and Measurement of Cathepsin K Calibration Curve

The experimental principle was basically the same as described in Example 1. A peptide (SEQ ID NO: 1) cleavable by cathepsin K was designed. The peptide had two functional groups, one was carboxyl group (—COOH), and another was amino group (—NH₂). The amino group had to be pre-protected to prevent the binding between peptides when carboxyl group was activated. 1.2 equivalents of fluorenylmethyloxycarbonyl chloride were added into 1 equivalent of peptide solution to protect the amino group of peptide. After reaction of 6 hours, 1.2 equivalents of N,N′-dicyclohexylcarbodiimide (DCC) solution were added to activate the carboxyl group. After reaction of 6 hours, GNRs modified with amino groups after centrifugation were added into the solution. After reaction of 1.5 days, the solution was centrifugated to collect the GNR product bonded with peptides. 20% piperidine in dimethyl formamide (DMF) was added into the product for reaction of 4 hours to remove the protective group fluorenylmethyloxycarbonyl chloride. The solution was then centrifugated to collect and purify deprotected GNRs bonded with peptides.

An appropriate amount of 1-Ethyl-3-(3-dimethylaminopropyl)carbodiimide (EDC) was added into the gold nanocluster solution. After reaction of 4 hours, 0.1 ml DMF and aforementioned deprotected GNRs bonded with peptides (on which the amino groups were exposed) were added. After reaction of 1.5 days, the gold nanoclusters bonded to the peptides on GNRs, forming all gold fluorescence probes. The all gold fluorescence probes were then collected by centrifugation and used in the following experiments.

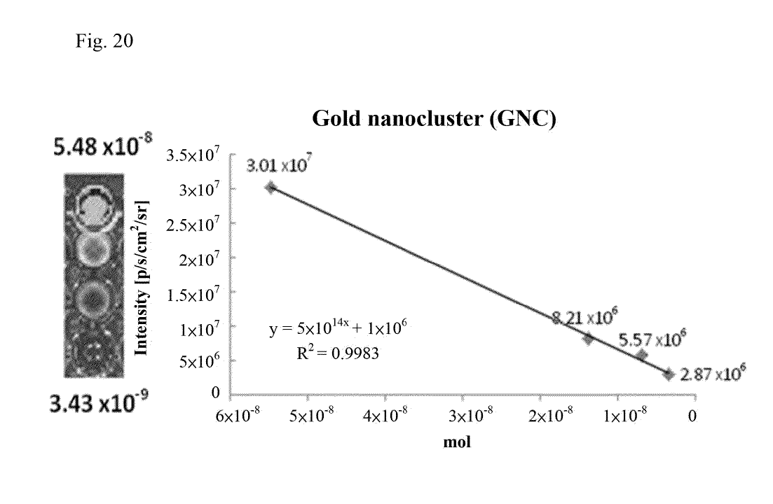

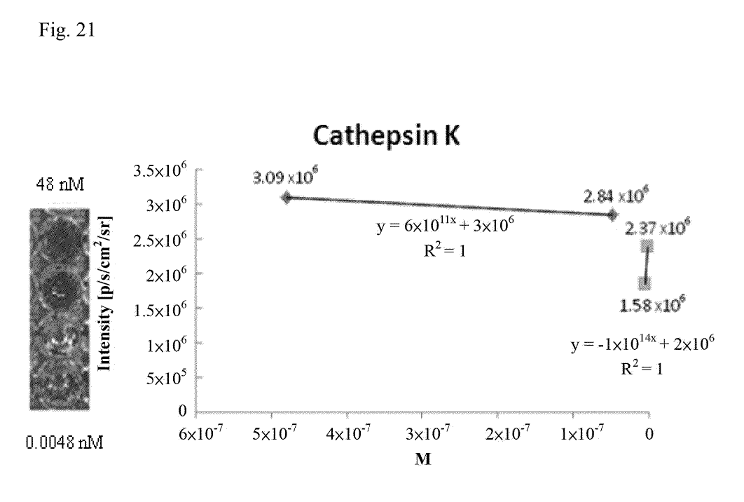

20 μL cathepsin K with different concentrations (48 nM, 4.8 nM, 0.48 nM, and 0.048 nM) were added into 40 μl all gold fluorescence probe in a 96-well plate for reaction of 2 hours in 37° C. water bath for making cathepsin K calibration curve. Further, 40 μl GNCs (54.8 nmol, 13.7 nmol, 6.85 nmol, and 3.43 nmol) were added into the 96-well plate for making GNC calibration curve. IVIS photos (Ex: 640 nm, Em: 760 nm) were taken to determine the fluorescence and make the GNC and cathepsin K calibration curves, shown in FIG. 20 and FIG. 21 respectively. The GNC curve showed a linear relationship (R²=0.998) (FIG. 20) while the cathepsin K curve showed a 2-stage linear relationship at higher concentration of 48 nM to 4.8 nM and lower concentration of 0.48 nM to 0.048 nM (FIG. 21). The cathepsin K fluorescence value was substituted into the GNC calibration curve formula y=5×10^{14x}+1×10⁶ to obtain the corresponding GNC amount (Table 1).

TABLE 1

| | | | | |
|---|---|---|---|---|
| Cathepsin K | 4.8 × 10⁻⁸ | 4.8 × 10⁻⁹ | 4.8 × 10⁻¹⁰ | 4.8 × 10⁻¹¹ |
| concentration (M) | | | | |
| GNC amount | 4.2 × 10⁻⁹ | 3.7 × 10⁻⁹ | 1.7 × 10⁻⁹ | 2.7 × 10⁻⁹ |
| (mol) | | | | |

In Vitro: Activity and Concentration of Cathepsin K from SD Rats with Arthritis Induced by Inflammatory Factor IL-1β were Investigated

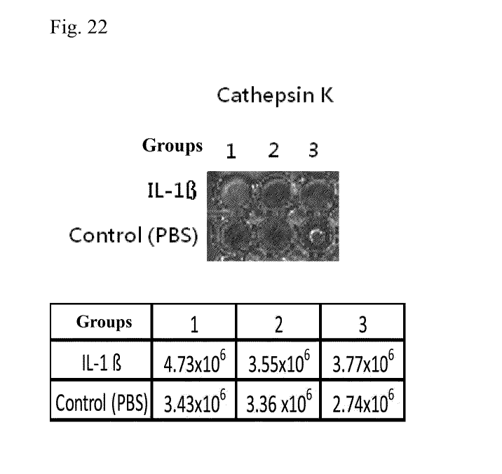

IL-1β is an inflammatory factor. IL-1β injection would make joints produce enzymes (cathepsin K, ADAMTS-4, MMP-13 . . . etc.) which would cause arthritis. Therefore, IL-1β could be used for investigating the activity of cathepsin K by detection of all gold fluorescence probes in vitro. Arthritis induced by IL-1β was investigated in the experiment. 40 μl IL-1β (1 μg) and 40 μl PBS (control) were injected into articular cavities of three SD rats (n=3), respectively. After 20 minutes, the synovial fluid was drawn out. 20 μL of the fluid was added into a 96-well plate and 40 μl of all gold fluorescence probes cleavable by cathepsin K was added for reaction of 2 hours in 37° C. water bath. The fluorescence was determined by IVIS imaging (Ex: 640 nm, Em: 760 nm). The detection result of the all gold fluorescence probe system was shown in FIG. 22. The IVIS result of the synovial fluid produced by IL-1β-induced SD rats proved that the all gold fluorescence probes had ability of detecting cathepsin K. The fluorescence values of IL-1β groups were substituted into formulas of cathepsin K and GNC calibration curves to obtain the cathepsin K concentration and GNC amounts (Table 2). For determining cathepsin K concentration, the fluorescence value ranging from 4.73×10⁶ to 3.36×10⁶ was substituted into the linear formula y=6×10^{11x}+3×10⁶, and the fluorescence value of 2.74×10⁶ was substituted into the linear formula y=−1×10^{14x}+2×10⁶. The fluorescence value was also substituted into GNC calibration curve formula y=5×10^{14x}+1×10⁶ to determine the GNC amount. The data showed that the group injected with inflammatory factor had higher fluorescence values compared to control group (without injection of inflammatory factor), which was resulted from cleavage of cathepsin K.

TABLE 2

| | IL-1β | | | Control (PBS) | | |
|---|---|---|---|---|---|---|
| Groups | 1 | 2 | 3 | 1 | 2 | 3 |
| Cathepsin K | 7.39 | 5.41 | 5.78 | 5.22 | 5.10 | 4.06 |
| concentration | | | | | | |
| (μM) | | | | | | |
| GNC amount | 7.47 | 5.10 | 5.53 | 4.86 | 4.71 | 3.48 |
| (nmol) | | | | | | |

In Vitro: Activity and Concentration of Cathepsin K from Nubian Goats with Arthritis were Investigated

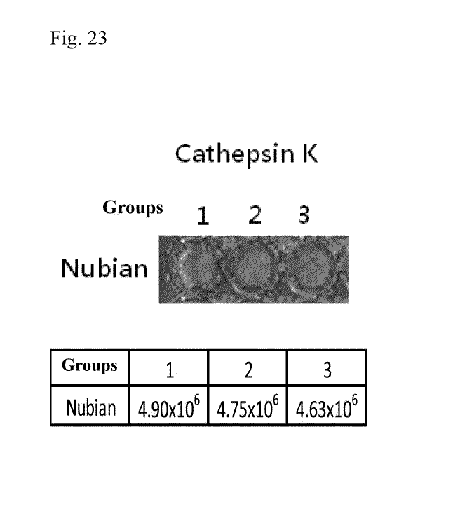

The Nubian goats' synovial fluid was collected for enzyme assessment in vitro. Arthritis usually occurred in goat knees. Therefore, this experiment was carried out to determine whether Nubian goats had idiopathic arthritis. Synovial fluid from Nubian goats (n=3) was drawn out. 20 μL of the synovial fluid was added into a 96-well plate and 40 μl of the all gold fluorescence probes cleavable by cathepsin K was added for reaction of 2 hours in 37° C. water bath. The fluorescence was determined by IVIS imaging (Ex: 640 nm, Em: 760 nm). The detection result of the all gold fluorescence probe system was shown in FIG. 23. The IVIS result of the synovial fluid from Nubian goats proved that the all gold fluorescence probes had ability of detecting cathepsin K. The fluorescence values were substituted into formulas of cathepsin K and GNC calibration curves to obtain the enzyme (cathepsin K) concentration and GNC amounts (Table 3). For determining cathepsin K concentration, the fluorescence value ranging from 4.90×10⁶ to 4.63×10⁶ was substituted into the linear formula y=6×10^{11x}+3×10⁶. The fluorescence value was also substituted into GNC calibration curve formula y=5×10^{14x}+1×10⁶ to determine the GNC amount. The data showed that the Nubian goat of group 1 had higher cathepsin K concentration than group 2 and group 3, indicating that Nubian goat of group 1 had more severe arthritis which led to a higher cathepsin K concentration.

TABLE 3

| Groups | 1 | 2 | 3 |
|---|---|---|---|
| Cathepsin K | 7.66 | 7.42 | 7.22 |
| concentration (μM) | | | |
| GNC amount (nmol) | 7.79 | 7.50 | 7.27 |

### Example 6

Non-Invasive In Vivo Imaging System (Caliper IVIS System, IVIS) for Assessment and Measurement of the Detection of all Gold Fluorescence Probe of the Present Invention against ADAMTS-4

The detection principle was the same as described in Example 5. Therefore, it would be more precise to place GNC sample (for making GNC calibration curve), ADAMTS-4 sample (for making ADAMTS-4 calibration curve) and biological sample in the same 96-well plate for assessment.

### Assessment and Measurement of ADAMTS-4 Calibration Curve

The experimental principle was basically the same as described in Example 1. A peptide (SEQ ID NO: 3) cleavable by ADAMTS-4 was designed. The peptide had two functional groups, one was carboxyl group (—COOH), and another was amino group (—NH₂). The amino group had to be pre-protected to prevent the binding between peptides when carboxyl group was activated. 1.2 equivalents of fluorenylmethyloxycarbonyl chloride were added into 1 equivalent of peptide solution to protect the amino group of peptide. After reaction of 6 hours, 1.2 equivalents of N,N′-dicyclohexylcarbodiimide (DCC) solution were added to activate the carboxyl group. After reaction of 6 hours, GNRs modified with amino groups after centrifugation were added into the solution. After reaction of 1.5 days, the solution was centrifugated to collect the GNR product bonded with peptides. 20% piperidine in dimethyl formamide (DMF) was added into the product for reaction of 4 hours to remove the protective group fluorenylmethyloxycarbonyl chloride. The solution was then centrifugated to collect and purify deprotected GNRs bonded with peptides.

An appropriate amount of 1-Ethyl-3-(3-dimethylaminopropyl)carbodiimide (EDC) was added into the gold nanocluster solution. After reaction of 4 hours, 0.1 ml DMF and aforementioned deprotected GNRs bonded with peptides (on which the amino groups were exposed) were added. After reaction of 1.5 days, the gold nanoclusters bonded to the peptides on GNRs, forming all gold fluorescence probes. The all gold fluorescence probes were then collected by centrifugation and used in the following experiments.

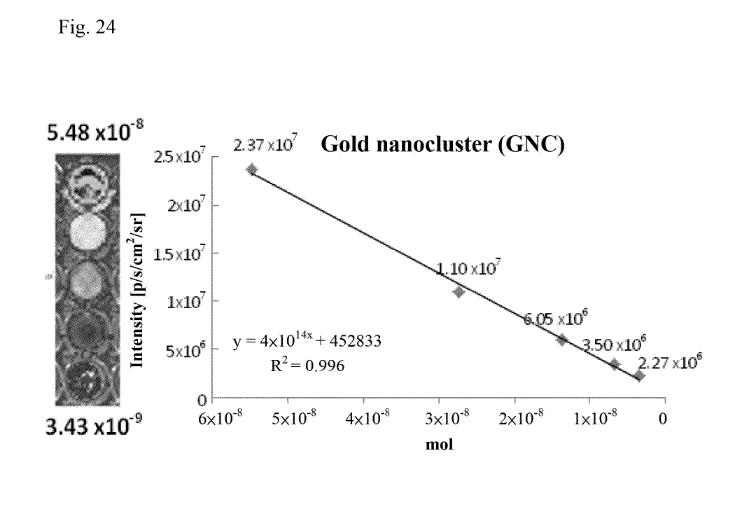

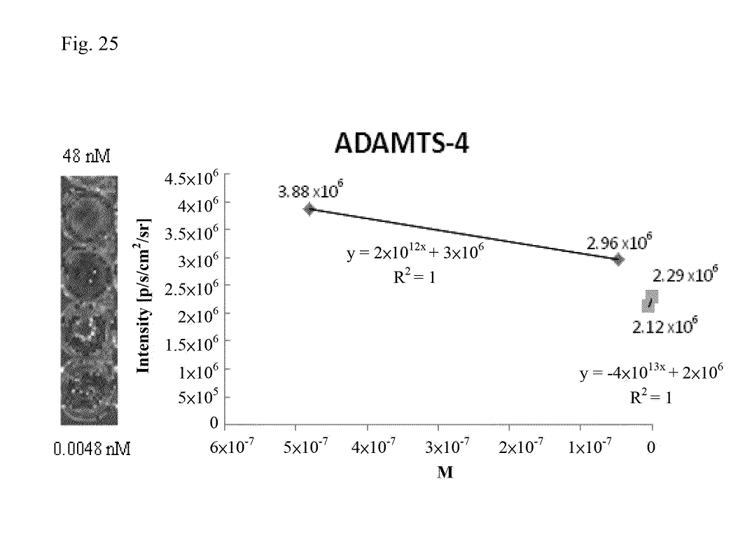

20 μL ADAMTS-4 with different concentrations (48 nM, 4.8 nM, 0.48 nM, and 0.048 nM) were added into 40 μl all gold fluorescence probe in a 96-well plate for reaction of 2 hours in 37° C. water bath for making ADAMTS-4 calibration curve. Further, 40 μl GNCs (54.8 nmol, 27.4 nmol, 13.7 nmol, 6.85 nmol, and 3.43 nmol) were added into the 96-well plate for making GNC calibration curve. IVIS photos (Ex: 640 nm, Em: 760 nm) were taken to determine the fluorescence and make the GNC and ADAMTS-4 calibration curves, shown in FIG. 24 and FIG. 25 respectively. The GNC curve showed a linear relationship (R²=0.996) (FIG. 24) while the ADAMTS-4 curve showed a 2-stage linear relationship at higher concentration of 48 nM to 4.8 nM and lower concentration of 0.48 nM to 0.048 nM (FIG. 25). The ADAMTS-4 fluorescence value was substituted into the GNC calibration curve formula y=4×10^{14x}+452833 to obtain the corresponding GNC amount (Table 4).

TABLE 4

| | | | | |
|---|---|---|---|---|
| ADAMTS-4 | 4.8 × 10⁻⁸ | 4.8 × 10⁻⁹ | 4.8 × 10⁻¹⁰ | 4.8 × 10⁻¹¹ |
| concentration | | | | |
| (M) | | | | |
| GNC amount | 8.57 | 6.27 | 4.17 | 4.59 |
| (nmole) | | | | |

In Vitro: Activity and Concentration of ADAMTS-4 from SD Rats with Arthritis Induced by Inflammatory Factor IL-1β were Investigated

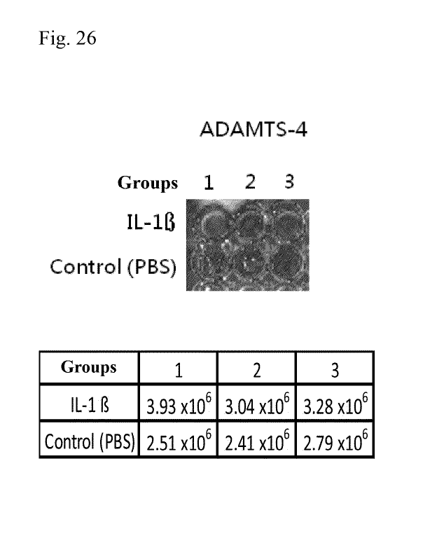

IL-1β is an inflammatory factor. IL-1β injection would make joints produce enzymes (cathepsin K, ADAMTS-4, MMP-13 . . . etc.) which would cause arthritis. Therefore, IL-1β could be used for investigating the activity of ADAMTS-4 by detection of all gold fluorescence probes in vitro. Arthritis induced by IL-1β was investigated in the experiment. 40 μl IL-1β (1 μg) and 40 μl PBS (control) were injected into articular cavities of three SD rats (n=3), respectively. After 20 minutes, the synovial fluid was drawn out. 20 μL of the fluid was added into a 96-well plate and 40 μl of all gold fluorescence probes cleavable by ADAMTS-4 was added for reaction of 2 hours in 37° C. water bath. The fluorescence was determined by IVIS imaging (Ex: 640 nm, Em: 760 nm). The detection result of the all gold fluorescence probe system was shown in FIG. 26. The IVIS result of the synovial fluid produced by IL-1β-induced SD rats proved that the all gold fluorescence probes had ability of detecting ADAMTS-4. The fluorescence values of IL-1β groups were substituted into formulas of ADAMTS-4 and GNC calibration curves to obtain the ADAMTS-4 concentration and GNC amounts (Table 5). For determining ADAMTS-4 concentration, the fluorescence value ranging from 93×10⁶ to 3.93×10⁶ (IL-1β groups) was substituted into the linear formula y=2×10^{12x}+3×10⁶, and the fluorescence value ranging from 2.51×10⁶ to 2.79×10⁶ (control groups) was substituted into the linear formula y=2×10^{12x}+3×10⁶. The fluorescence value was also substituted into GNC calibration curve formula y=4×10^{14x}+452833 to determine the GNC amount. The data showed that the group injected with inflammatory factor had higher fluorescence values compared to control group (without injection of inflammatory factor), which was resulted from cleavage of ADAMTS-4.

TABLE 5

| | IL-1β | | | Control (PBS) | | |
|---|---|---|---|---|---|---|
| Groups | 1 | 2 | 3 | 1 | 2 | 3 |
| ADAMTS-4 | 46.6 | 220 | 14.1 | −127 | −102 | −199 |
| concentration | | | | | | |
| (μM) | | | | | | |
| GNC amount | 8.70 | 6.48 | 7.07 | 5.14 | 4.89 | 5.85 |
| (nmol) | | | | | | |

The data showed that detection accuracy was lower when the enzyme (ADAMTS-4) concentration was lower than 0.48 nM. It was presumed that this concentration was beyond the detection limit of the all gold fluorescence probe so that the enzyme concentration calculated by the formula was negative.

In Vitro: Activity and Concentration of ADAMTS-4 from Nubian Goats with Arthritis were Investigated

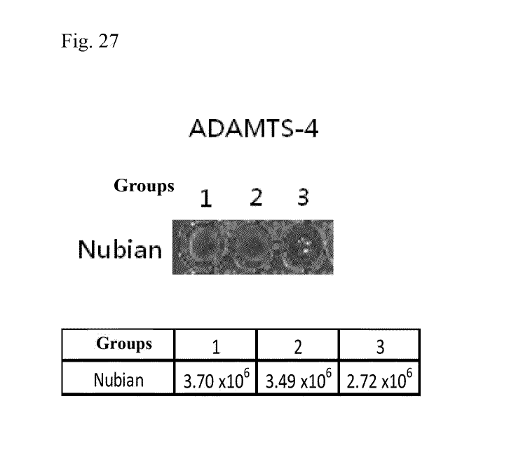

The Nubian goats' synovial fluid was collected for enzyme assessment in vitro. Arthritis usually occurred in goat knees. Therefore, this experiment was carried out to determine whether Nubian goats had idiopathic arthritis. Synovial fluid from Nubian goats (n=3) was drawn out. 20 μL of the synovial fluid was added into a 96-well plate and 40 μl of the all gold fluorescence probes cleavable by ADAMTS-4 was added for reaction of 2 hours in 37° C. water bath. The fluorescence was determined by IVIS imaging (Ex: 640 nm, Em: 760 nm). The detection result of the all gold fluorescence probe system was shown in FIG. 27. The IVIS result of the synovial fluid from Nubian goats proved that the all gold fluorescence probes had ability of detecting ADAMTS-4. The fluorescence values were substituted into formulas of ADAMTS-4 and GNC calibration curves to obtain the enzyme (ADAMTS-4) concentration and GNC amounts (Table 6). For determining ADAMTS-4 concentration, the fluorescence value ranging from 3.70×10⁶ to 2.72×10⁶ was substituted into the linear formula y=2×10^{12x}+3×10⁶. The fluorescence value was also substituted into GNC calibration curve formula y=4×10^{14x}+452833 to determine the GNC amount. The data showed that the Nubian goat of group 1 had higher ADAMTS-4 concentration than group 2 and group 3, indicating that Nubian goat of group 1 had more severe arthritis which led to a higher ADAMTS-4 concentration.

TABLE 6

| Groups | 1 | 2 | 3 |
|---|---|---|---|
| ADAMTS-4 | 35.2 | 24.4 | 13.9 |
| concentration | | | |
| (μM) | | | |
| GNC amount (nmol) | 8.13 | 7.59 | 5.68 |

### Example 7

Non-Invasive In Vivo Imaging System (Caliper IVIS System, IVIS) for Assessment and Measurement of the Detection of all Gold Fluorescence Probe of the Present Invention against MMP-13

The detection principle was the same as described in Example 5. Therefore, it would be more precise to place GNC sample (for making GNC calibration curve), MMP-13 sample (for making MMP-13 calibration curve) and biological sample in the same 96-well plate for assessment.

### Assessment and Measurement of MMP-13 Calibration Curve

The experimental principle was basically the same as described in Example 1. A peptide (SEQ ID NO: 4) cleavable by MMP-13 was designed. The peptide had two functional groups, one was carboxyl group (—COOH), and another was amino group (—NH₂). The amino group had to be pre-protected to prevent the binding between peptides when carboxyl group was activated. 1.2 equivalents of fluorenylmethyloxycarbonyl chloride were added into 1 equivalent of peptide solution to protect the amino group of peptide. After reaction of 6 hours, 1.2 equivalents of N,N′-dicyclohexylcarbodiimide (DCC) solution were added to activate the carboxyl group. After reaction of 6 hours, GNRs modified with amino groups after centrifugation were added into the solution. After reaction of 1.5 days, the solution was centrifugated to collect the GNR product bonded with peptides. 20% piperidine in dimethyl formamide (DMF) was added into the product for reaction of 4 hours to remove the protective group fluorenylmethyloxycarbonyl chloride. The solution was then centrifugated to collect and purify deprotected GNRs bonded with peptides.

An appropriate amount of 1-Ethyl-3-(3-dimethylaminopropyl)carbodiimide (EDC) was added into the gold nanocluster solution. After reaction of 4 hours, 0.1 ml DMF and aforementioned deprotected GNRs bonded with peptides (on which the amino groups were exposed) were added. After reaction of 1.5 days, the gold nanoclusters bonded to the peptides on GNRs, forming all gold fluorescence probes. The all gold fluorescence probes were then collected by centrifugation and used in the following experiments.

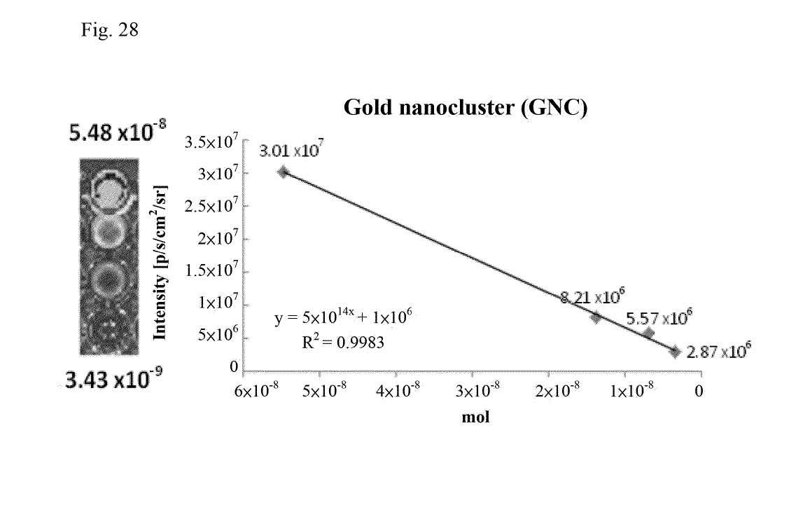

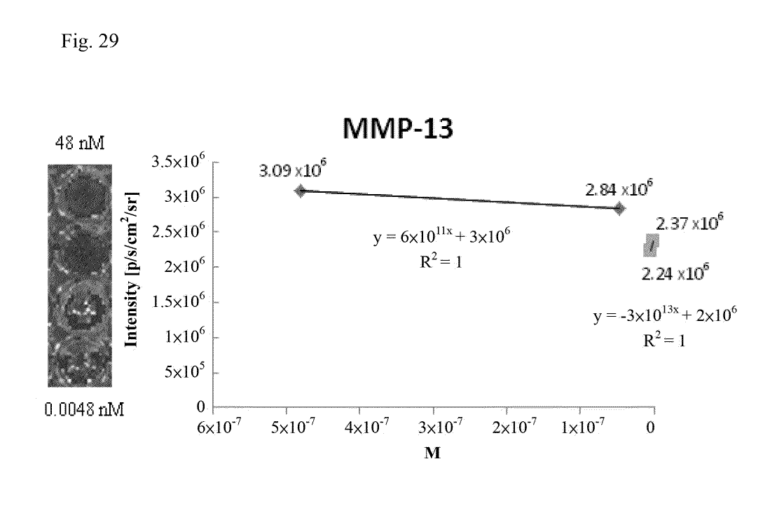

20 μL MMP-13 with different concentrations (48 nM, 4.8 nM, 0.48 nM, and 0.048 nM) were added into 40 μl all gold fluorescence probe in a 96-well plate for reaction of 2 hours in 37° C. water bath for making MMP-13 calibration curve. Further, 40 μl GNCs (54.8 nmol, 13.7 nmol, 6.85 nmol, and 3.43 nmol) were added into the 96-well plate for making GNC calibration curve. IVIS photos (Ex: 640 nm, Em: 760 nm) were taken to determine the fluorescence and make the GNC and MMP-13 calibration curves, shown in FIG. 28 and FIG. 29 respectively. The GNC curve showed a linear relationship (R²=0.998) (FIG. 28) while the MMP-13 curve showed a 2-stage linear relationship at higher concentration of 48 nM to 4.8 nM and lower concentration of 0.48 nM to 0.048 nM (FIG. 29). The MMP-13 fluorescence value was substituted into the GNC calibration curve formula y=5×10^{14x}+1×10⁶ to obtain the corresponding GNC amount (Table 7).

TABLE 7

| | | | | |
|---|---|---|---|---|
| MMP-13 | 4.8 × 10⁻⁸ | 4.8 × 10⁻⁹ | 4.8 × 10⁻¹⁰ | 4.8 × 10⁻¹¹ |
| concentration (M) | | | | |
| GNC amount | 4.18 | 3.68 | 2.48 | 2.74 |
| (nmol) | | | | |

In Vitro: Activity and Concentration of MMP-13 from SD Rats with Arthritis Induced by Inflammatory Factor IL-1β were Investigated

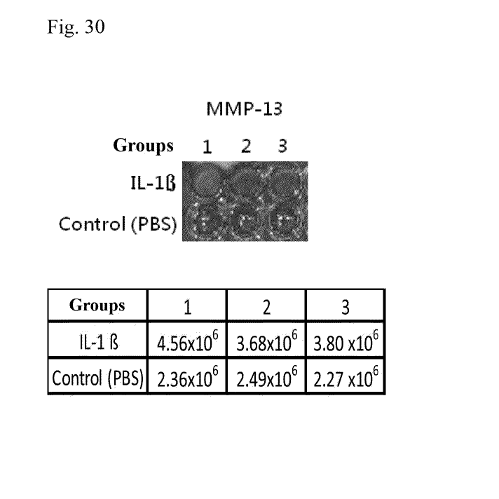

IL-1β is an inflammatory factor. IL-1β injection would make joints produce enzymes (cathepsin K, ADAMTS-4, MMP-13 . . . etc.) which would cause arthritis. Therefore, IL-1β could be used for investigating the activity of MMP-13 by detection of all gold fluorescence probes in vitro. Arthritis induced by IL-1β was investigated in the experiment. 40 μl IL-1β (1 μg) and 40 μl PBS (control) were injected into articular cavities of three SD rats (n=3), respectively. After 20 minutes, the synovial fluid was drawn out. 20 μL of the fluid was added into a 96-well plate and 40 μl of all gold fluorescence probes cleavable by MMP-13 was added for reaction of 2 hours in 37° C. water bath. The fluorescence was determined by IVIS imaging (Ex: 640 nm, Em: 760 nm). The detection result of the all gold fluorescence probe system was shown in FIG. 30. The IVIS result of the synovial fluid produced by IL-1β-induced SD rats proved that the all gold fluorescence probes had ability of detecting MMP-13. The fluorescence values of IL-1β groups were substituted into formulas of MMP-13 and GNC calibration curves to obtain the MMP-13 concentration and GNC amounts (Table 8). For determining MMP-13 concentration, the fluorescence value ranging from 3.68×10⁶ to 4.56×10⁶ (IL-1β groups) was substituted into the linear formula y=6×10^{11x}+3×10⁶, the fluorescence value of 2.49×10⁶ (control group 2) was substituted into the linear formula y=6×10^{11x}+3×10⁶, and the fluorescence value ranging from 2.27×10⁶ to 2.36×10⁶ (control groups 1 and 3) was substituted into the linear formula y=−3×10^{13x}+2×10⁶. The fluorescence value was also substituted into GNC calibration curve formula y=5×10^{14x}+1×10⁶ to determine the GNC amount. The data showed that the group injected with inflammatory factor had higher fluorescence values compared to control group (without injection of inflammatory factor), which was resulted from cleavage of MMP-13.

TABLE 8

| | IL-1β | | | Control (PBS) | | |
|---|---|---|---|---|---|---|
| Groups | 1 | 2 | 3 | 1 | 2 | 3 |
| MMP-13 | 2.60 | 1.14 | 1.33 | −1.08 | −84.5 | −9000 |
| concentration | | | | | | |
| (μM) | | | | | | |
| GNC amount | 7.12 | 5.37 | 5.59 | 2.71 | 2.99 | 2.54 |
| (nmol) | | | | | | |

The data showed that detection accuracy was lower when the enzyme (MMP-13) concentration was lower than 0.48 nM. It was presumed that this concentration was beyond the detection limit of the all gold fluorescence probe so that the enzyme concentration calculated by the formula was negative.

In Vitro: Activity and Concentration of MMP-13 from Nubian Goats with Arthritis were Investigated

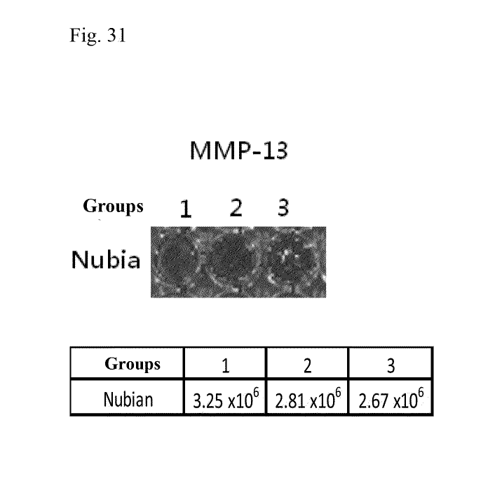

The Nubian goats' synovial fluid was collected for enzyme assessment in vitro. Arthritis usually occurred in goat knees. Therefore, this experiment was carried out to determine whether Nubian goats had idiopathic arthritis. Synovial fluid from Nubian goats (n=3) was drawn out. 20 μL of the synovial fluid was added into a 96-well plate and 40 μl of the all gold fluorescence probes cleavable by MMP-13 was added for reaction of 2 hours in 37° C. water bath. The fluorescence was determined by IVIS imaging (Ex: 640 nm, Em: 760 nm). The detection result of the all gold fluorescence probe system was shown in FIG. 31. The IVIS result of the synovial fluid from Nubian goats proved that the all gold fluorescence probes had ability of detecting MMP-13. The fluorescence values were substituted into formulas of MMP-13 and GNC calibration curves to obtain the enzyme (MMP-13) concentration and GNC amounts (Table 9). For determining MMP-13 concentration, the fluorescence value ranging from 2.67×10⁶ to 3.25×10⁶ was substituted into the linear formula y=6×10^{11x}+3×10⁶. The fluorescence value was also substituted into GNC calibration curve formula y=5×10^{14x}+1×10⁶ to determine the GNC amount. The data showed that the Nubian goat of group 1 had higher MMP-13 concentration than group 2 and group 3, indicating that Nubian goat of group 1 had more severe arthritis which led to a higher MMP-13 concentration.

TABLE 9

| Groups | 1 | 2 | 3 |
|---|---|---|---|
| MMP-13 | 37.8 | −32.3 | −224 |
| concentration (μM) | | | |
| GNC amount (nmol) | 4.45 | 3.61 | 3.35 |

The data showed that detection accuracy was lower when the enzyme (MMP-13) concentration was lower than 0.48 nM. It was presumed that this concentration was beyond the detection limit of the all gold fluorescence probe so that the enzyme concentration calculated by the formula was negative.

According to the above examples, it is proved that the all gold fluorescence probe of the present invention has good cleavage efficiency for detecting the three enzymes cathepsin K, ADAMTS-4, and MMP-13. Therefore, the all gold fluorescence probe of the present invention has potential to be developed as a simple, fast and effective disease detection tool in the future.

One skilled in the art readily appreciates that the present invention is well adapted to carry out the objects and obtain the ends and advantages mentioned, as well as those inherent therein. The gold nanoprobes, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A gold fluorescence resonance energy transfer nanoprobe comprising a gold fluorescence donor, a gold fluorescence acceptor, and a linker fragment that connects the gold fluorescence donor and the gold fluorescence acceptor, wherein the fluorescence resonance energy transfer is carried out between the gold fluorescence donor and the gold fluorescence acceptor.

2. The gold fluorescence resonance energy transfer nanoprobe of claim 1, wherein the linker fragment is a peptide.

3. The gold fluorescence resonance energy transfer nanoprobe of claim 2, wherein the peptide has a length of 3 to 20 amino acids.

4. The gold fluorescence resonance energy transfer nanoprobe of claim 2, wherein the peptide is cleaved by an enzyme so that the fluorescence resonance energy transfer between the gold fluorescence donor and the gold fluorescence acceptor no longer occurs.

5. The gold fluorescence resonance energy transfer nanoprobe of claim 4, wherein the activity or concentration of the enzyme in a patient or a nidus is higher than in a normal subject or normal tissue.

6. The gold fluorescence resonance energy transfer nanoprobe of claim 5, wherein the enzyme is matrix metalloproteinase, cathepsin, or aggrecanase.

7. The gold fluorescence resonance energy transfer nanoprobe of claim 5, wherein the nidus is an arthritis site.

8. The gold fluorescence resonance energy transfer nanoprobe of claim 1, wherein the gold fluorescence acceptor is a gold nanorod.

9. The gold fluorescence resonance energy transfer nanoprobe of claim 8, wherein the gold nanorod has a length of 50 to 200 nm.

10. The gold fluorescence resonance energy transfer nanoprobe of claim 8, wherein the absorption wavelength of the gold nanorod ranges from 470 to 570 nm and/or 600 to 950 nm.

11. The gold fluorescence resonance energy transfer nanoprobe of claim 1, wherein the gold fluorescence donor is a fluorescence gold nanocluster.

12. The gold fluorescence resonance energy transfer nanoprobe of claim 11, wherein the fluorescence gold nanocluster has a length of 5 to 50 nm.

13. The gold fluorescence resonance energy transfer nanoprobe of claim 11, wherein the emission wavelength of the fluorescence gold nanocluster ranges from 600 to 950 nm.

14. The gold fluorescence resonance energy transfer nanoprobe of claim 1, which is used for in vivo or in vitro detection.

15. The gold fluorescence resonance energy transfer nanoprobe of claim 1, which is used in X-ray contrast imaging.
